(19) United States
(12) Patent Application Publication
(10) Pub. No.: US 2020/0029858 A1
(43) Pub. Date: Jan. 30, 2020

(54) Systems, Methods and Devices for Detecting and Identifying Substances in a Subject's Breath

(71) Applicant: nGagelT Digital Health, Inc., Toronto (CA)

(72) Inventor: Raj Reddy, Toronto (CA)

(21) Appl. No.: 16/357,097

(22) Filed: Mar. 18, 2019

Related U.S. Application Data
(60) Provisional application No. 62/711,603, filed on Jul. 29, 2018.

(51) Int. Cl. A61B 5/08; G01N 33/00; G01N 33/497; A61B 5/00; A61B 5/097

(52) U.S. Cl. CPC A61B 5/082; G01N 33/0047; G01N 33/497; G01N 2033/4975; A61B 5/097; A61B 5/0022; A61B 5/7264

(57) ABSTRACT

Embodiments of the disclosure can include systems, methods, and devices for detecting and identifying certain substances, such as volatile organic compounds (VOCs), volatile gases (VGs), and ketones, in the exhaled breath of a subject or person in real-time.

### RELATED APPLICATION

This application claims priority under 35 U.S.C. § 119(e) from U.S. Provisional Application Ser. No. 62/711,603, filed Jul. 28, 2018, which is hereby incorporated by reference in its entirety.

### TECHNICAL FIELD

The disclosure relates to detection and identification of certain substances in the exhaled breath of a subject or person, and in particular to systems, methods, and devices for detecting and identifying certain substances, such as volatile organic compounds (VOCs), volatile gases (VGs), and ketones, in the exhaled breath of a subject or person in real-time.

### BACKGROUND

Human breath is mainly composed of nitrogen, oxygen, carbon dioxide, water vapor, and inert gases. In addition, thousands of volatile organic compounds (VOCs) may be exhaled at very low concentrations. The metabolism of fat, in particular the breakdown of triglycerides, can lead to the accumulation of ketone bodies in a person's blood. These ketone bodies can include acetone, acetoacetic acid, and beta-hydroxybutyric acid. In the blood, acetone exists in the form of acetoacetate. Further, during periods of restricted calorie input, the concentration of ketone bodies and fatty acids can increase, whereas the concentration of glucose may fall.

Digestive cancers belong to the most widespread and deadly human cancers. The digestive system includes any number of organs and body parts including, but not limited to, the esophagus, stomach, small intestine, colon, rectum, anus, liver, pancreas, gallbladder and biliary system. Colorectal cancer and stomach cancer, for example, are the second leading causes of cancer deaths in the United States and worldwide, respectively. Early pre-symptomatic detection is paramount in the management of digestive cancers, thus improving prognosis and treatment outcome.

Further, sepsis is a serious health condition that is caused by a subject's body response to an infection. The body's immune system typically releases various chemicals into the subject's blood to combat the infection, but the subject's body may adversely react with severe and sometimes fatal consequences.

Controlled substances can generally be any legal or illicit consumable drug, chemical, or other substance which is controlled by a government regulation, and, in certain jurisdictions, can include cannabis and alcohol. Detection of cannabis and other controlled substances in a person can be invasive in nature, and are commonly performed by urine, blood, or oral specimen sampling. Conventional specimen sampling can be relatively sophisticated, and may require a complex device for analysis. In one example, alcohol can be examined directly by a conventional exhaled breath exam, most commonly by exhaling into an ion spectroscopy chamber. While this conventional solution has proven reliable and is accepted by legal systems as a non-invasive method to quantify alcohol levels, testing for other controlled substances, such as cannabis, generally lacks a similar solution. The terms “controlled substance(s)” and “drug(s)” are used interchangeably throughout this disclosure.

Further, weight control is a goal of a relatively large proportion of the U.S. population. Conventional weight control programs typically allow a restricted range of caloric intake per day, with some allowance made for activity levels. However, even though caloric intake is monitored with some precision, the effects of physical activity are not measured in a quantitative way. Physical activity can be a relatively important component of weight control programs for several reasons. First, physical activity can be used to reduce the body fat proportion of a person. Next, physical activity can help reduce the fall in resting metabolic rate of a person on a restricted caloric intake. Physical activity is initially fueled by blood sugar, but after a sustained period of activity a person will start to metabolize fat. Few people on weight control programs are aware of how much exercise is required to start the fat metabolizing process, and they may not be fully aware of the beneficial effects of activity on their resting metabolic rate.

### BRIEF DESCRIPTION

Some or all of the above needs and/or problems may be addressed by certain embodiments of the disclosure. Certain embodiments can include systems, methods, and devices for detecting and identifying certain substances, such as chemicals, volatile organic compounds (VOCs), volatile gases (VGs), ketones, cannabis, controlled substances, or pharmaceuticals in the exhaled breath of a subject or person in real-time.

According to one embodiment, there is disclosed a system for detecting and identifying one or more volatile organic compounds (VOCs) in exhaled breath of a subject. In this embodiment, the system can include a mouth piece connected to a housing, the mouth piece operable to receive the exhaled breath of the subject; a sensor module disposed in the housing, the sensor module operable to detect the one or more VOCs in the exhaled breath, and further operable to collect data associated with detection of the one or more one or more VOCs; and a communication module disposed in the housing and in communication with the sensor module, the communication module operable to transmit collected data from the sensor module to a remote processor for analysis of the collected data.

In at least one aspect of the embodiment, the sensor module can include at least one sensor component operable to detect one or more VOCs, wherein the sensor component includes at least one of the following: an electronic sensor, an electromechanical sensor, an electrochemical sensor, a nanoparticle sensor, a 2D (two-dimensional) metal carbide, a 2D (two-dimensional) Ti3C2 nanosheet, a MXene; a customized form 2D Mn+iXn (Ts) MXene composition; a 2D (two-dimensional) Ti3C2 nanosheet; Ti3C2Tx; Ti₃C₂(OH)₂; Ti₃C₂ MXene; a 3D MXene (3D-M) framework; one or more metal oxide nanoparticles blended with a hybrid structure of graphene; a conducting polymer such as polyaniline (PAni) and polypyrrole (PPY); a supramolecule; a cavitand (macrocyclic compounds based on resorcinarenes); or a relatively thin film of a supramolecule or a calixresorcinarene.

In at least one aspect of the embodiment, the sensor module can include at least one array of respective sensors, wherein each sensor is operable to detect at least one VOC.

In at least one aspect of the embodiment, the VOCs can include at least one of the following: a ketone, acetone, an aldehyde, an acetaldehyde, a hydrocarbon, an alkane, a pentane, an alkene, or a fatty acid.

In at least one aspect of the embodiment, the one more VOCs can be associated with a health condition including at least one of the following: diabetes, high or low blood glucose levels, ketoacidosis, lung cancer, breast cancer, a digestive cancer, gastric cancer, peptic ulcer, colorectal cancer, prostate cancer, head-and-neck cancer, stomach cancer, liver cancer, kidney disease, or neurodegenerative disease.

In at least one aspect of the embodiment, the system can include a biomarker processing module, in communication with the sensor module, and operable to process the collected data associated with detection of the one or more VOCs and to identify the one or more VOCs.

In at least one aspect of the embodiment, the biomarker processing module is further operable to process the collected data via a neural network or pattern recognition algorithm, wherein a result from the biomarker processing module is received by the communication module for output to a mobile communication device associated with the subject.

In at least one aspect of the embodiment, the biomarker processing module is further operable to process the collected data in conjunction with other sensor data, wherein a result from the biomarker processing module is received by the communication module for output to a mobile communication device associated with the subject.

In at least one aspect of the embodiment, the system can include an activity sensor operable to detect or measure one or more physical actions by the subject, and further operable to collect data associated with detection or measurement of the one or more physical actions; and wherein the communication module is in communication with the activity sensor, and the communication module is further operable to transmit collected data from the activity sensor.

In at least one aspect of the embodiment, the communication module is further operable to communicate with a mobile communication device associated with the subject, wherein the mobile communication device receives the collected data from the sensor module and the collected data from the activity sensor.

In at least one aspect of the embodiment, the communication module is further operable to transmit collected data via at least one of the following: IR (infrared) communication, wireless communication, a Bluetooth protocol wireless communication, a direct wired connection, or to a remote memory storage device.

According to another embodiment of the disclosure, a method can be provided. The method can include receiving an exhaled breath of the subject; detecting, via a nanoparticle sensor, one or more VOCs in the exhaled breath; based at least in part on detection of the one or more VOCs, generating an electronic signal associated with a concentration or amount of the one or more VOCs; and outputting, via a display device, an indication of a health condition or disease associated with the concentration or amount of the one or more VOCs.

In at least one aspect of the embodiment, the method can include determining the electronic signal correlates with a predefined signal or signal pattern associated with a health condition or disease; and identifying, based at least in part on the determination of a correlation with a predefined signal or signal pattern, a health condition or disease.

In at least one aspect of the embodiment, the method can include processing the electronic signal via a neural network or pattern recognition algorithm, wherein the electronic signal correlates with a predefined signal or signal pattern associated with a health condition or disease; and identifying, based at least in part on the determination of a correlation with a predefined signal or signal pattern, a health condition or disease.

In at least one aspect of the embodiment, the method can include classifying the electronic signal as a new signal or signal pattern associated with the health condition or disease; and storing the new pattern in a data storage device.

In at least one aspect of the embodiment, the method can include facilitating a treatment for the subject to address the health condition or disease.

According to another embodiment of the disclosure, a sensor can be provided. The sensor can include a first sensor component operable to expose one or more nanoparticles to at least one VOC (volatile organic compound) in an exhaled breath of a subject, wherein the one or more nanoparticles are operable to react to a presence of or contact with the at least one VOC (volatile organic compound); a second sensor component operable to generate an electronic signal when the one or more nanoparticles react to the presence of or contact with the at least one VOC, wherein the electronic signal is associated with a concentration or amount of the at least one VOC; and an electronic circuit operable to transmit the electronic signal to an output device or computer processor.

In at least one aspect of the embodiment, the first sensor component can include at least one of the following: a 2D (two-dimensional) metal carbide, a 2D (two-dimensional) Ti3C2 nanosheet, a MXene; a customized form 2D Mn+iXn (Ts) MXene composition; a 2D (two-dimensional) Ti3C2 nanosheet; Ti3C2Tx; Ti₃C₂(OH)₂; Ti₃C₂ MXene; a 3D MXene (3D-M) framework; one or more metal oxide nanoparticles blended with a hybrid structure of graphene; a conducting polymer such as polyaniline (PAni) and polypyrrole (PPY); a supramolecule; a cavitand (macrocyclic compounds based on resorcinarenes); or a relatively thin film of a supramolecule or a calixresorcinarene.

In at least one aspect of the embodiment, the VOCs can include at least one of the following: a ketone, acetone, an aldehyde, an acetaldehyde, a hydrocarbon, an alkane, a pentane, an alkene, or a fatty acid.

In at least one aspect of the embodiment, wherein the one or more nanoparticles include a plurality of different nanoparticles, each of the plurality of different nanoparticles selected to trap, bind, or react with a respective different VOC, and wherein the electronic signal includes respective signal components associated with each of the plurality of different nanoparticles trapping, binding, or reacting with a respective different VOC.

Other embodiments, systems, methods, devices, aspects, and features of the disclosure will become apparent to those skilled in the art from the following detailed description, the accompanying drawings, and the appended claims.

### BRIEF DESCRIPTION OF THE FIGURES

The detailed description is set forth with reference to the accompanying drawings, which are not necessarily drawn to scale. The use of the same reference numbers in different figures indicates similar or identical items.

FIG. 1 depicts an architectural view of an example system and device for detecting certain substances including VOCs, VGs, and ketones, according to one example embodiment of the disclosure.

FIG. 2 depicts a schematic view of an example sensor and associated sensor components for detecting certain substances including VOCs, VGs, and ketones, according to one example embodiment of the disclosure.

FIG. 3 depicts another schematic view of an example sensor and associated sensor components for detecting certain substances including VOCs, VGs, and ketones, according to one example embodiment of the disclosure.

FIG. 4 depicts a schematic view of an example sensor and associated sensor components for detecting certain substances including VOCs, VGs, and ketones, according to one example embodiment of the disclosure.

FIGS. 5 and 6 depict an example analysis of collected data for a health condition or disease, according to one example embodiment of the disclosure.

FIG. 7 depicts an example method for detecting certain substances including VOCs, VGs, and ketones, according to one example embodiment of the disclosure.

FIG. 8 depicts an example heat map graphic generated by a biomarker processing module or engine, or diagnostic module, according to one example embodiment of the disclosure.

FIG. 9 depicts another example heat map graphic generated by a biomarker processing module or engine, or diagnostic module, according to one example embodiment of the disclosure.

Certain implementations will now be described more fully below with reference to the accompanying drawings, in which various implementations and/or aspects are shown. Various aspects may, however, be implemented in many different forms and should not be construed as limited to the implementations set forth herein. Like numbers refer to like elements throughout. The following detailed description includes references to the accompanying drawings, which form part of the detailed description. The drawings depict illustrations, in accordance with example embodiments. These example embodiments, which are also referred to herein as “examples,” are described in enough detail to enable those skilled in the art to practice the present subject matter. The example embodiments may be combined, other embodiments may be utilized, or structural, logical, and electrical changes may be made, without departing from the scope of the claimed subject matter. The following detailed description is, therefore, not to be taken in a limiting sense, and the scope is defined by the appended claims and their equivalents.

### DETAILED DESCRIPTION

Illustrated embodiments herein are directed to systems, methods, and devices for detecting and identifying certain substances, such as volatile organic compounds (VOCs), volatile gases (VGs), and ketones, in the exhaled breath of a subject or person in real-time. Further, certain embodiments of the disclosure can be directed to systems, methods, and devices for exercise monitoring and diet management of a subject or person. Technical effects of certain embodiments of the disclosure may include providing diagnosis and treatment for particular health conditions related to the detection and identification of certain substances, such as volatile organic compounds (VOCs), volatile gases (VGs), and ketones, in the exhaled breath of a subject or person in real-time. Further, technical effects of certain embodiments of the disclosure may include providing real-time evaluation of an exercise and/or diet regimen prescribed for a subject or person.

Various conventional technologies and techniques have previously been implemented to measure certain substances in a subject including U.S. Pat. No. 4,114,422 to Hutson (acetone); U.S. Pat. Nos. 4,758,521 and 4,970,172 to Kundu (ketones and acetone); U.S. patent application Ser. No. 09/630,398 and International Application WO2001/093743A2 to Mault et al. (oxygen, ketones); U.S. Pat. Nos. 4,931,404; 4,970,172; 5,071,769; 5,174,959; and 5,834,626 to De Castro et al. (ketones); U.S. Pat. No. 5,996,586 to Phillips (ketones, aldehydes, hydrocarbons, and alkenes, fatty acids); International Application WO2017180606A1 to Atsalakis (oxygen, carbon dioxide); and U.S. Pat. No. 9,562,915 to Burgi (alcohol, acetone, NO, H2, HN3), but none of these technologies or techniques address the specific needs that various embodiments of the disclosure address. Each of these prior references is hereby incorporated by reference.

Novel sensor technology, such as those utilizing nanoparticles and nanomaterials, can be combined with mobile communication devices, such as smart phones, and cloud computing to create technical solutions for respiratory analysis, diagnosis, and subsequent treatment. A nanoparticle sensor used in combination with the processor of a smart phone and/or remote server, and a biomarker processing module or engine with a neural network or pattern matching algorithm, can be used to detect VOCs (volatile organic compounds), volatile gases (VGs), and ketones, exhaled by persons or mammals in their breath. The detection and measurement of VOCs (volatile organic compounds), volatile gases (VGs), and ketones, can be specifically correlated with certain health conditions or disease, such as cancer, lung cancer, breast cancer, digestive cancers, diabetes, ulcers, peptic ulcers, sepsis, or levels of exhaled anesthetic gases used in medical procedures for surgery. Real-time breath testing by simply exhaling into a device attached to a smart phone can provide particularly useful technical results, because the detection and measurement data can be immediately processed by the smart phone or a remote processor via a cloud computing service, and a biomarker processing module or engine, and then real-time results can be made available to a clinician, physician, or other professional, thus permitting relatively fast diagnoses and corresponding treatment decisions. Embodiments of the disclosure can have many useful and valuable applications in the industrial and biomedical industries, food and utility industries, health care and medical care sectors, and security services.

As used herein, the term “health condition” refers to any condition affecting or afflicting a body of a human or mammal. Health conditions that can be detected by certain embodiments of the disclosure can include, for example, but not limited to, cancer, ulcers, diabetes, acute asthma, hepatic coma, rheumatoid arthritis, schizophrenia, ketosis, cardiopulmonary disease, uremia, diabetes mellitus, dysgeusia/dysosmia, cystinuria, cirrhosis, histidinemia, tyrosinemia, halitosis, and phenylketonuria, etc.

As used herein, the term “cancer” refers to a disorder in which a population of cells has become, in varying degrees, unresponsive to the control mechanisms that normally govern proliferation and differentiation. Cancer refers to various types of malignant neoplasms and tumors, including metastasis to different sites. Examples of cancers which can be detected by certain embodiments of the disclosure can include, but are not limited to, brain, ovarian, colon, prostate, kidney, bladder, breast, lung, oral, and skin cancers. Specific examples of cancers are: adenocarcinoma, adrenal gland tumor, ameloblastoma, anaplastic tumor, anaplastic carcinoma of the thyroid cell, angiofibroma, angioma, angiosarcoma, apudoma, argentaffinoma, arrhenoblastoma, ascites tumor cell, ascitic tumor, astroblastoma, astrocytoma, ataxia-telangiectasia, atrial myxoma, basal cell carcinoma, benign tumor, bone cancer, bone tumor, brainstem glioma, brain tumor, breast cancer, vaginal tumor, Burkitt's lymphoma, carcinoma, cerebellar astrocytoma, cervical cancer, cherry angioma, cholangiocarcinoma, a cholangioma, chondroblastoma, chondroma, chondrosarcoma, chorioblastoma, choriocarcinoma, larynx cancer, colon cancer, common acute lymphoblastic leukemia, craniopharyngioma, cystocarcinoma, cystofibroma, cystoma, cytoma, ductal carcinoma in situ, ductal papilloma, dysgerminoma, encephaloma, endometrial carcinoma, endothelioma, ependymoma, epithelioma, erythroleukaemia, Ewing's sarcoma, extra nodal lymphoma, feline sarcoma, fibroadenoma, fibrosarcoma, follicular cancer of the thyroid, ganglioglioma, gastrinoma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angiofibroma, Kaposi sarcoma, kidney tumor, large cell lymphoma, leukemia, chronic leukemia, acute leukemia, lipoma, liver cancer, liver metastases, Lucke carcinoma, lymphadenoma, lymphangioma, lymphocytic leukaemia, lymphocytic lymphoma, lymphocytoma, lymphoedema, lymphoma, lung cancer, malignant mesothelioma, malignant teratoma, mastocytoma, medulloblastoma, melanoma, meningioma, mesothelioma, metastatic cancer, Morton's neuroma, multiple myeloma, myeloblastoma, myeloid leukemia, myelolipoma, myeloma, myoblastoma, myxoma, nasopharyngeal carcinoma, nephroblastoma, neuroblastoma, neurofibroma, neurofibromatosis, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheochromocytoma, plasmacytoma, primary brain tumor, progonoma, prolactinoma, renal cell carcinoma, retinoblastoma, rhabdomyosarcoma, rhabdosarcoma, solid tumor, sarcoma, secondary tumor, seminoma, skin cancer, small cell carcinoma, squamous cell carcinoma, strawberry haemangioma, T-cell lymphoma, teratoma, testicular cancer, thymoma, trophoblastic tumor, tumourigenic, vestibular schwannoma, Wilm's tumor, or a combination thereof.

As used herein, “biomarker” refers to one or more signals and/or signal patterns associated with a presence of one or more substances, concentrations and/or amounts of respective substances associated with diagnosing, treating, or addressing a health condition or disease. In some instances, a biomarker can refer to one or more signals and/or signal patterns associated with a presence of a specific combination of substances at predefined concentrations and/or amounts.

As used herein, “real-time” refers to an event or a sequence of acts, such as those executed by a computer processor that are perceivable by a subject, person, user, or observer at substantially the same time that the event is occurring or that the acts are being performed. By way of example, if a neural network receives an input based on sensing and identifying an exhaled gas, a result can be generated at substantially the same time that the exhaled gas was sensed and identified. The real-time processing of the input by the neural network may have a slight time delay associated with converting the sensed exhaled gas to an electrical signal for an input to the neural network; however, any such delay may typically be less than 1 minute and usually no more than a few seconds.

One skilled in the art will recognize that various embodiments of the disclosure discuss the analysis of exhaled gases, though certain embodiments of the disclosure can also be used for the analysis of inhaled gases and the monitoring of pollutants or environmental effects. These embodiments may be useful when monitoring the administration of gases to a subject, such as for anesthetics, nitric oxide, oxygen, medications, or other treatments as well as for diagnoses and treatments of subjects respiring with the assistance of a ventilator or for subjects using a breathing apparatus.

### Example Detection/Identification Systems, Devices, Methods

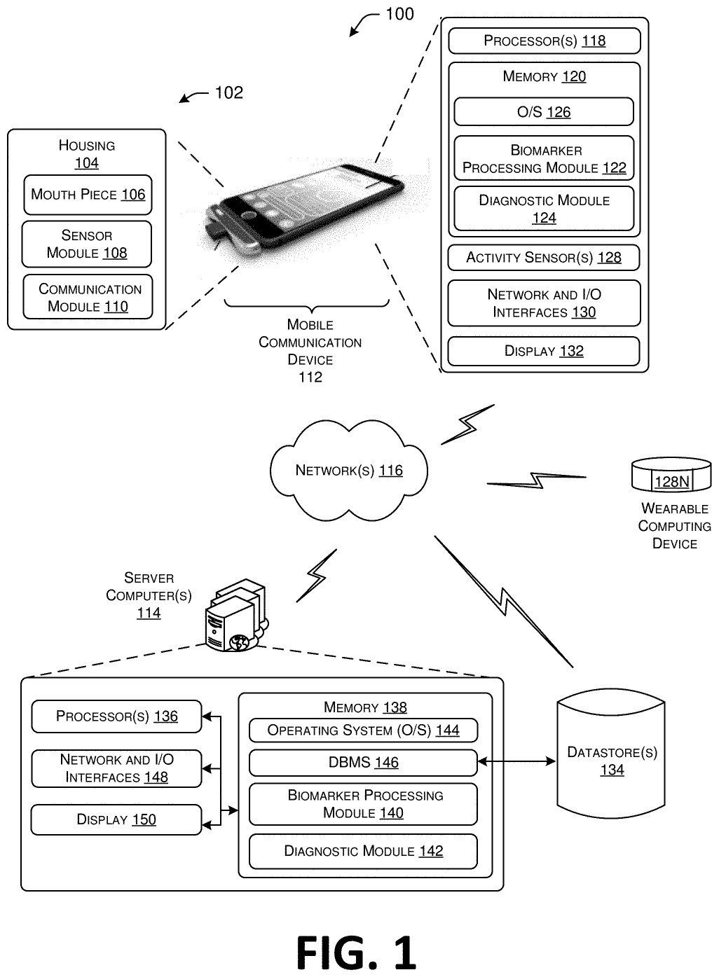

FIG. 1 depicts an example system 100 and device 102 for detecting and identifying certain substances in an exhaled breath of a subject. The example device 102, as shown in FIG. 1, can include a housing 104 which includes a mouth piece 106, a sensor module 108, and a communication module 110. The device 102 can be a handheld structure mounted to a smart phone or could be a stand alone structure, which as needed could be a portable structure.

As used herein, the term “substance” can include any VOC, VG, non-VOC, non-VG, chemical, element, or compound. Example substances can include, but are not limited to, chemicals, VOCs, VGs, ketones, acetone, aldehydes, aromatic aldehydes, benzaldehyde, alkanes, aromatic alkanes, ketons, 2-nonanone, 4-methyl-2-heptanone, 2-dodecanone, methanol, ethanol, secondary alcohol, cyclohexanol, fatty alcohol, 2-ethylhexanol, carboxylic acid, isobutyric acid, allyl ester, hydrocarbons, 2,3-dimethylhexane, 2-4-dimethyl-1-heptene, 2,2-dimethylbutane, 1,3-dis-ter-butylbenzene, 2-xylene, aromatic amines, pyrrolidine, 2-propenenitrile, 2-butoxy-ethanol, furfural, 6-methyl-5-hepten-2-one, isoprene decane, benzene, non-VOCs, ammonia, cannabis, controlled substances, pharmaceuticals, medications, anesthetics, propafol, nitric oxide, carbon monoxide, and carbon dioxide, among others. Other substances may include respiration components produced by certain bacteria within a subject's mouth, stomach, and/or intestinal tract. In addition, substances can include other bodily fluids and secretions from a subject including, but not limited to, serum, urine, feces, sweat, vaginal discharge, saliva, and sperm.

Generally, the housing 104 can be operable to mount to or otherwise can communicate with a mobile communication device 112, such as a smart phone, tablet, laptop, computer, a wearable computing device, a smart watch, a wearable activity tracker, or other wireless communication or computing device. In the example shown if FIG. 1, the housing 104 can mount to a communication and/or power port of the mobile communication device 112, wherein the communication module 110 can facilitate communications between the housing 104 and the mobile communication device 112 via the communication and/or power port. Examples of suitable communication and/or power ports can include, but are not limited to, Lightning, USB, micro-USB, serial, etc. In another example, the housing may adhere to or otherwise may be relatively close proximity to the mobile communication device 112, wherein the communication module 110 can facilitate communications between the housing 104 and the mobile communication device 112 via a wireless communication technique or protocol, such as IR (infrared) communication, cellular communication, Bluetooth, or WiFi.

In any instance, the housing 102 can include a mouth piece 106 operable to receive an exhaled breath from a subject, such as a person or mammal. For example, a person can insert the mouth piece 106 in his or her mouth, closing his or her lips around the mouth piece 106, and the person can breathe into the mouth piece, thus exhaling his or her breath into the mouth piece 106. The mouth piece 106 can include an inlet opening to receive the exhaled breath from the subject, and can further include an adjacent flow path, wherein the exhaled breath can, when received from the mouth piece 106, travel towards the sensor module 108.

In at least one embodiment, the mouth piece 106 can be operable to permit a subject to inhale and exhale into the housing 104 without adversely or substantially affecting the one or more samples of exhaled breath from the subject.

In at least one embodiment, a housing, such as 104, can include a sensor chamber disposed between the mouth piece 106 and a first end. For example, the sensor chamber may be a concentric-shaped chamber surrounding the flow path between the mouth piece 106 and the first end. A ketone sensor, for instance, may be positioned within the sensor chamber, wherein the ketone sensor, upon detection of a ketone in an exhaled breath within the flow path, can generate a signal or signal pattern correlated with a ketone concentration and/or amount in the exhaled breath.

In at least one embodiment, a housing, such as 104, can include a drying mechanism or technique, wherein the exhaled breath can be dried or excess moisture in the exhaled breath can be removed. For example, the housing 104 can include a silica gel to remove moisture from the exhaled breath. In any instance, the drying mechanism or technique should maintain substantial proportions of any gas component of interest or otherwise not affect the amount of gases in the exhaled breath of the subject.

In at least one embodiment, a housing, such as 104, can include a flow regulator operable to admit a sufficient sample volume of exhaled breath into the housing 104.

In at least one embodiment, a housing, such as 104, can include a power charger connection. For example, to facilitate mounting the housing 104 to a communication and/or power port of a mobile communication device 112, the housing can include a power charger connection, which may include both power and communication connections to exchange power and communications with the mobile communication device 112. In some embodiments, power and communication connections between the housing 104 and mobile communication device 112 may be separate connections and/or ports. In another example, the housing 104 may include a rechargeable battery, which can provides power to the various functionality of the housing 104 and associated device, and a power charger connection may facilitate charging the rechargeable battery, while the communication module 110 facilitates communications with the mobile communication device 112 by a wireless communication protocol or technique.

In the example shown in FIG. 1, the sensor module 108 can be operable to detect certain substances, such as, for instance, one or more VOCs, in the exhaled breath, and further operable to collect data associated with detection of the substances. The sensor module 108 can include at least one sensor component operable to detect at least one substance, for instance, a VOC, VG, or ketone. Depending on the specific substance to be detected and/or identified, any number of sensors and/or sensor components can be selected and/or designed to provide suitable sensitivity for detecting and identifying the specific substance as well as providing for suitable detection and/or identification of a relative concentration or amount of the specific substance. Suitable sensors and sensor components can include, but are not limited to, electronic sensors, electromechanical sensors, electrochemical sensors, or any other sensing device or technique that can convert detection of certain substances in the exhaled breath to an electrical signal.

In at least one embodiment, a sensor module, such as 108, can be an electronic gas sensing device ranging from about 10-100 nanometers to a few micrometers in dimension, and could be installed in any handheld electronic and/or mobile communication device. For example, a sensor module, such as 108, could be integrated in a smart watch or cellular phone.

In any instance, the one or more substances, can be detected and identified by the sensor module 108. Thus, when the exhaled breath is received from the mouth piece 106, the exhaled breath flows through, over, or otherwise adjacent to at least one sensor or sensor component of the sensor module 108, which can detect and identify one or more substances in the exhaled breath. In the embodiment shown in FIG. 1, the sensor module 108 can, upon detection and identifying one or more substances, in the exhaled breath, generate an electronic signal correlating to a relative concentration or amount of a specific substances detected or otherwise identified in the exhaled breath. In some embodiments, any number of electronic signals may be generated by a sensor module 108 depending on the number of detected and identified substances. Each of the electronic signals would correlate to a relative concentration or amount of a specific substance detected or otherwise identified in the exhaled breath of the subject.

In at least one embodiment, a sensor module, such as 108, can be customized to detect any number of volatile gas (VG) patterns identified in the one or more VOCs of the exhaled breath of the subject. For example, if a predefined VG pattern including 3 specific VOCs with respective threshold concentrations is associated with a health condition or disease, a sensor module, such as 108, can be customized or otherwise designed to include one or more sensors operable to detect and identify the 3 specific VOCs as well as detect and identify a concentration and/or amount of the respective VOCs. The predefined volatile gas (VG) patterns can be specifically correlated with a particular health condition and/or disease, such as lung cancer, breast cancer, and/or digestive cancers, or otherwise correlated with levels of exhaled anesthetic gases used for medical or surgical procedures. Different health conditions and diseases, such as cancers, emit different types and/or amounts of molecules. For example, endogenous cancer may release certain VOCs from the cancer cells and/or metabolic processes that are associated with cancer growth may release similar or other VOCs. In any case, these VOCs can be transported with a subject's blood to the alveoli of the subject's lungs from where they can be exhaled as measurable smells or odorants. Therefore, cancer not only has a characteristic smell or odor, but, different cancers can have different and unique smells or odors. When one or more signals and/or signal patterns of a certain number and quantity (by concentration and/or amount) of substances is associated with a particular health condition or disease, the one or more signals and/or signal patterns can be stored by the system 100 for subsequent processing to compare and/or match against signals and/or signal patterns associated with substances previously detected and identified by a sensor module, such as 108, in any number of respective exhaled breaths of any number of subjects.

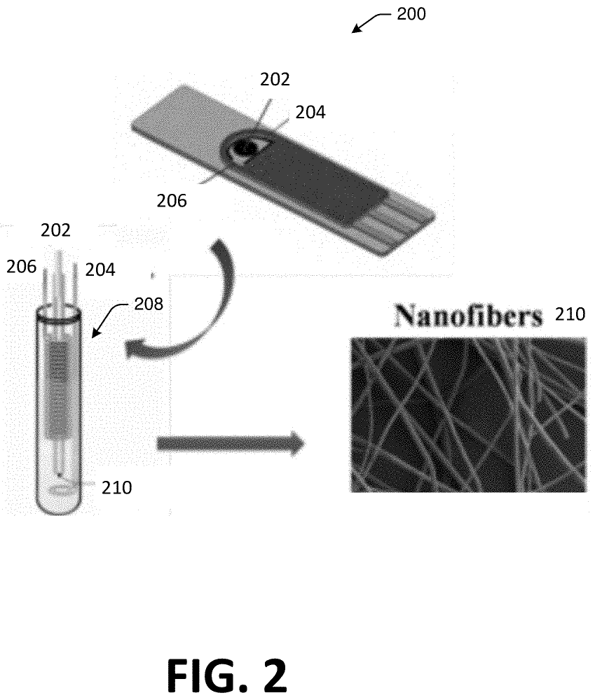

In at least one embodiment, ketones, such as acetone, can be detected and/or identified by a sensor module, such as 108, which can include a sensor, or sensor component, such as 200 shown in FIG. 2, with one or more nanomaterials. Any type of nanomaterials for detecting ketones, such as acetone, can be used including nanocomposites, nanotubules, or nanofibers. The sensor component 200 shown in FIG. 2 can include a working electrode 202, a counter electrode 204, and a reference electrode 206. When the sensor component 200 is assembled with other components to form a sensor 208, the counter electrode 204 can be made from a conducting paint which can be carbon paint. The working electrode 202 can be made from a conducting paint and can be connected to a bed of carbon nanofibers or carbon nanofibers with multi-walled carbon nanotubules. The reference electrode 206 can be made from a conducting paint such as a silver (Ag) material. An electrode cross section 210 can be fabricated from a bed of carbon nanofibers 212, which can be embedded with sensing enhancing nanoparticles. Generally, the signal response generated by a sensor component, such as 200, may be dependent on the electrochemical characteristics of molecules of the substance that are either oxidized or reduced at the working electrode, such as 202, with the opposite occurring at the counter electrode, such as 204. The resulting voltage generated by the differential reactions between the electrodes, such as 202 and 204, can be measured, and with respect to the reference electrode, such as 206, can be used to generate an electronic signal and/or signal pattern from the sensor component 200.

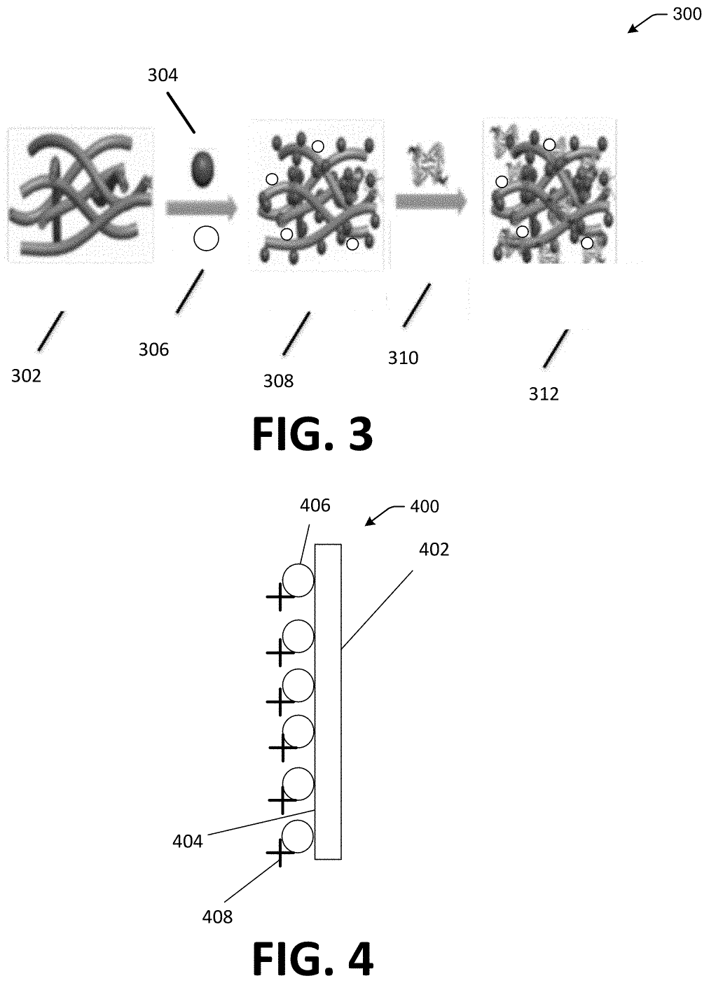

In at least one embodiment, certain substances, such as VOCs, can be detected and identified using a sensor module, such as 108, which can include a sensor or sensor component, such as 300 shown in FIG. 3, with one or more nanofibers, such as polymeric nanofibers, conducting polymers or, for example, carbon nanofibers (CNF) 302. The carbon nanofibers (CNF) 302 shown in this example can be embedded with selective nanoparticles, such as nanometal oxide (MOX) 304. In some embodiments, the CNF 302 can be enhanced with one or more other selective molecules 306, such as particles, compositions, supramolecules, cavitands, etc. In any instance, a CNF/MOX matrix or enhanced CNF/MOX, such as 308, can be formed. When the exhaled breath of a subject includes one or more VOCs, such as acetone 310, is passed through, over, or adjacent to the CNF/MOX matrix or enhanced CNF/MOX matrix 308, the MOX 304 and/or one or more other selective molecules 306 can selectively trap, as shown in 312, one or more VOCs including acetone. For example, the MOX 304 can bond, trap, or otherwise selectively react with a first VOC, such as acetone 310, and the other selective molecules 306 can bond, trap, or otherwise selectively react with a second VOC. In this manner, the resulting trapped VOC 310 and/or other VOCs within the MOX 304 and/or one or more other selective molecules 306 of the CNF/MOX matrix or enhanced CNF/MOX matrix 308 can facilitate generating an electronic signal and/or signal pattern from the sensor 300, the signal and/or signal pattern corresponding in strength to a concentration and/or amount of the VOCs, such as 310, trapped within the MOX 304 and/or one or more other selective molecules 306 of the CNF/MOX matrix or enhanced CNF/MOX matrix 308. The signal and/or signal pattern can be transmitted to the communication module 110, and further transmitted to the mobile communication device 112 for subsequent processing.

By way of example, suitable materials and structures for electrodes in a sensor module, such as 108, can include, but are not limited to, graphene, gold nanowires, nanocomposites, nanotubules, and nanofibers.

By way of example, a nanoparticle can be defined as a solid colloidal particle having a size in the range from about 10 nm (nanometers) to about 1000 nm, the size of which can offer many benefits to relatively larger particles such as increased surface-to-volume ratio and increased magnetic properties. Generally, a nanoparticle surface can, at a molecular level, adsorb or otherwise selectively trap one or more substances upon or adjacent to the nanoparticle surface. Suitable materials and structures for nanoparticles in a sensor module, such as 108, can include, but are not limited to, iron oxide nanoparticles (which can be selected for their relative chemical stability, non-toxicity, biocompatibility, high saturation magnetization, and high magnetic susceptibility), mesoporuous silica nanoparticles, superpapramagnetic nanoparticles, core-shell structure nanoparticles (inorganic-inorganic, inorganic-organic, organic-inorganic and organic-organic such as PLA-PEG), silver nanoparticles, and gold nanoparticles. One skilled in the art will recognize the various methods and techniques for the synthesis of nanoparticles including, but not limited to, using a top-down approach (mechanical attrition, nanolithography, etching, physical vapor deposition (PVD), chemical vapor deposition (CVD)), and bottom-up approach (sol-gel, solvothermal, sonochemical method, microwave-assisted synthesis, reduction in solution, template synthesis, co-precipitation, biosynthesis). Characteristics for suitable nanoparticles used in a sensor module, such as 108, can include, but are not limited to, sensitivity, reliability, adjustability of chemical and physical properties to detect and identify certain substances, such as VOCs and/or VG (volatile gas) patterns, in relatively humid atmospheres, ease of fabrication, and cost effective production.

In at least one embodiment, the sensor module 108 can include one or more nanocomposites, nanotubules, or nanofibers with any number of immobilized substances embedded or otherwise mounted within them, such as biological enzymes (e.g., laccase) or custom nanoparticles, which can be tuned to detect and/or identify any number of specific gases and/or substances exhaled in the breath of a subject. For example, when exhaled breath including one or more substances, such as VOCs including ketones or aldehydes, are passed through, over, or adjacent to the sensor or sensor component, the one or more immobilized substances can selectively trap one or more substances. The reaction between the trapped substances and the immobilized substances and/or nanocomposites, nanotubules, or nanofibers can, similar to the example embodiment depicted in FIG. 3, facilitate generating an electronic signal and/or signal pattern from the sensor module 108. The signal and/or signal pattern corresponding in strength to a concentration and/or amount of the trapped substances, such as VOCs, within the one or more immobilized substances. The signal and/or signal pattern can be transmitted to a communication module, such as 110, and further transmitted to a mobile communication device, such as 112, for subsequent processing.

FIG. 4 depicts a schematic view of another example sensor and associated sensor components, according to one example embodiment of the disclosure. The example sensor 400 and sensor components can incorporated into a sensor module, similar to the sensor module 108, and can operate in conjunction with and in a similar manner as the system 100 and device 102 shown in FIG. 1. In the embodiment shown in FIG. 4, a sensor 400 and sensor components, can include an electrode 402 with at least one surface 404, a set of nanoparticles 406, and a set of target antibodies 408. In one example, the sensor, such as 400, can be an electrode covered with gold nanoparticles with immobilized antibodies. When an exhaled breath of a subject flows over, adjacent, or through the sensor, a substance within the exhaled breath can bind to the immobilized antibodies. The combination of the substance in the exhaled breath and the antibodies can generate an electrical signal via the gold nanoparticles 406 on the electrode 402. The signal can be transmitted from the sensor 400 or sensor component to a communication module, such as 110 in FIG. 1, and processed by a biomarker processing module 122 or 140, in a similar manner by the system 100 and device 102 shown in FIG. 1.

In at least one embodiment, an electrode, such as 402, can be any suitable material for immobilizing and/or attracting any number of nanoparticles to at least one surface of the electrode.

In at least one embodiment, one or more nanoparticles, such as 406, can include, but are not limited to, gold, metal, or non-metal nanoparticles.

In at least one embodiment, one or more target antibodies, such as 408, can include, but are not limited to, antibodies operable to change an electrical property or otherwise generate an electrical signal when in contact with a predefined substance such as a VOC, VG, or ketone.

One skilled in the art will recognize that a sensor, such as 400, can utilize any number of sensor components and/or materials that may provide suitable sensitivity and detection characteristics for any number of substances such as a VOC, VG, or ketone.

One skilled in the art will recognize that any number, types, and materials can be used for suitable electrodes, nanoparticles, and target antibodies, according to various embodiments of the disclosure. Further, any number of different operating architectures implementing one or more electrodes, nanoparticles, and target antibodies, according to various embodiments of the disclosure, can be used for an example sensor, similar to 400, and/or sensor components incorporated into a sensor module, similar to 108, and may operate in conjunction with and in a similar manner as the system 100 and device 102 shown in FIG. 1.

One skilled in the art will recognize that electrochemical sensors generally contain one or more electrodes and at least one electrolyte. The response generated by such sensors are usually dependent on the electrochemical characteristics of the volatile molecules that are oxidized or reduced at one or more working electrodes, with the opposite occurring at one or more counter electrodes. The voltage generated by the reactions between the working electrodes and counter electrodes can be measured, and the resulting electrical signal and/or signal patterns can be detected and identified.

In at least one embodiment, a sensor module, such as 108, can include one or more selectively permeable membranes to permit certain gases in the exhaled breath, such as nitrogen, oxygen, and/or carbon dioxide to exit the housing 104 while concentrating or otherwise retaining one or more substances, for detection and/or identification by the sensor module 108. For example, silicone rubber-type membranes can be used to separate certain gases from mixtures of nitrogen, oxygen, and/or carbon dioxide and other gases.

In at least one embodiment, a saliva trap device or technique can be incorporated into the device 102. In certain instances, saliva contamination of the exhaled breath may be of concern, and the saliva trap device or technique can minimize such concerns.

By way of example, in at least one embodiment, a sensor module, such as 108, can include at least one of the following: an electrochemical sensor; a chemiresistor, a chemicapacitor, a nanoparticle sensor; a 2D (two-dimensional) metal carbide/nitride such as a MXene; customized forms 2D Mn+iXn (Ts) MXene compositions; a 2D (two-dimensional) Ti3C2 nanosheet; Ti3C2Tx; Ti₃C₂(OH)₂; Ti₃C₂ MXene; a 3D MXene (3D-M) framework; one or more metal oxide nanoparticles blended with a hybrid structure of graphene; a conducting polymer such as polyaniline (PAni) and polypyrrole (PPY); a supramolecule; a cavitand (macrocyclic compounds based on resorcinarenes); or a relatively thin film of a supramolecule or a calixresorcinarene.

In at least one embodiment, a sensor module, such as 108, can include at least one sensor with at least one 2D (two-dimensional) metal carbide such as MXene or a 2D (two-dimensional) Ti3C2 nanosheet. Generally, 2D (two-dimensional) metal carbides such as MXenes can have a relatively high metallic conductivity to provide relatively low noise, and a highly receptive surface to generate a relatively strong electrical signal. MXenes can, in some instances, be organized into a 3D MXene (3D-M) framework or structure, which can be made with an electrospinning technique coupled with a self-assembly approach. In this manner, an example sensor module with a MXene in a 3D-M framework can perform at room temperature, can display a relatively high sensitivity to extremely low concentrations (ranging from several parts per million (ppm) to several parts per billion (ppb) levels) of certain substances, such as, for instance, VOCs, due to an interconnected porous structure, which can enable relatively easy access and diffusion of gas molecules within the structure. MXene materials are generally good at both allowing gas molecules to move across their surface, and such materials are also good at snagging, or adsorbing, certain substances that are chemically attracted to it, thus demonstrating relatively good selectivity. Furthermore, an example sensor module with a MXene in a 3D-M framework can exhibit an ultra-wide sensing range (from about 50 ppb and up, for certain saturated vapors, to several ppm, for certain VOCs), relatively fast response and recovery (less than about 2 minutes), and relatively good reversibility and flexibility (no performance decrease for about 1000 bending cycles) for certain substances, such as, for instance, VOCs (e.g., acetone, methanol, and ethanol). An example sensor module with a MXene in a 3D-M framework can be suitable for both industrial and biomedical applications, including food and utility industries, the health care and medical care sectors, and security services. Thus, according to various embodiments of the disclosure, a sensor module, such as 108, incorporating MXene in a 3D-M framework can be operable to generate a signal and/or signal pattern associated with the detection and/or identification of a particular substance associated with a specific health condition or disease. In some instances, a sensor incorporating MXene in a 3D-M framework can be tuned to generate a signal and/or signal pattern associated with the detection and/or identification of a predefined threshold, concentration and/or amount of a particular substance associated with a specific health condition or disease. In any instance, according to certain embodiments of the disclosure, the use and implementation of incorporating MXene in a 3D-M framework in a sensor can provide selective and targeted ultra-high sensitivity to one or more substances.

In at least one embodiment, a sensor module, such as 108, can include Ti₃C₂ MXene to detect and identify various VOCs, VGs, and ketones in an exhaled breath of a subject. A sensor module with a Ti₃C₂ MXene can be operable to detect a predefined threshold of at least 100 ppm ammonia, acetone, methanol, and ethanol gas at room temperature. Ti₃C₂ MXene can be synthesized by selective removal of Al from Ti₃AlC₂ MAX phase using LiF salt and HCl acid. A Ti₃C₂-based gas sensor can be fabricated on a flexible polyimide film via simple solution method. During at least one gas sensing test, Ti₃C₂-based gas sensor demonstrated initial resistance at room temperature at about 10˜20 kΩ with about 8 μm film thickness, and further demonstrated p type gas sensing behavior to each of ammonia, acetone, methanol, and ethanol gases. Such gas sensors can exhibit a very low limit of detection of about 50-100 ppb for certain VOC gases at room temperature. Also, the extremely low noise led to a signal-to-noise ratio about two orders of magnitude higher than that of conventional 2D materials. In order to obtain optimum high sensitivity in these gas sensors, two criteria are sought to be simultaneously satisfied: (1) low electrical noise induced by high conductivity, and (2) high signal induced by strong and abundant analyte adsorption sites.

In at least one embodiment, a sensor module, such as 108, can include Ti₃C₂T MXene film as metallic channels for chemiresistive gas sensors to detect and identify various VOCs, VGs, and ketones in an exhaled breath of a subject. Generally, MXenes are a family of 2D transition metal carbides/nitrides, where representative MXenes such as Ti₃C₂(OH)₂ possess metallic conductivity, while the outer surface is fully covered with functional groups, wherein metallic conductivity and abundant surface functionalities may coexist without mutual interference. Such a combination renders MXenes highly attractive for gas sensors with a high signal-to-noise ratio (SNR), which indicates the relative gas signal intensity over noise intensity, as the high coverage of functional groups allow strong binding with certain analytes, while the high metallic conductivity intrinsically leads to a low noise.

In at least one embodiment, a sensor module, such as 108, can include at least one sensor with at least one conducting polymer. Generally, conducting polymers can change their physical properties during reactions with various reduction agents. For example, suitable conducting polymers for a sensor module, such as 108, can include polyaniline (PAni) and polypyrrole (PPY). The relative resistivity of polypyrrole can increase in the presence of a reducing gas such as ammonia, but resistivity decreases in the presence of an oxidizing gas such as nitrogen dioxide. In this example, these gases can cause a change in the near-surface charge-carrier density of the conducting polymer, here polypyrrole, by reacting with surface-adsorbed oxygen ions. These types of changes in physical properties can permit one or more conducting polymers to be incorporated into a sensor operable to generate a signal and/or signal pattern associated with the detection and/or identification of a particular substance associated with a specific health condition or disease. In some instances, a sensor with one or more conducting polymers can be tuned to generate a signal and/or signal pattern associated with the detection and/or identification of a predefined threshold, concentration and/or amount of a particular substance associated with a specific health condition or disease. In any instance, according to certain embodiments of the disclosure, the use and implementation of one or more conducting polymers in a sensor can provide selective and targeted ultra-high sensitivity to one or more substances.

In at least one embodiment, a sensor module, such as 108, can include at least one sensor with at least one supramolecule or cavitand. Generally, supramolecules or cavitands can exhibit discotic phases depending on the particular structure of the supramolecule or cavitan, and can be used in certain applications where relatively high sensing is desireable. Further, relatively thin films of supramolecules or cavitands, such as calixresorcinarene, crown ethers, and calixarenes, can form inclusion complexes with certain organic guest molecules, and can be used in certain applications for recognizing organic vapors where relatively high sensing at room temperature is desireable. In one example, two cavitands, which have different sizes and shapes of a pre-organized cavity can be exposed to a variety of aromatic and chlorinated hydrocarbons. One cavitand, NHDxCav-1, can exhibit a marked preference for certain aromatic compounds with a sequence of selectivity, such as for nitrobenzene, toluene, and benzene. Another cavitand, NHDMeCav-2, can exhibit relatively higher selectivity for dichloromethane with respect to aromatic vapors at room temperature. Thus, the use and implementation of at least one supramolecule or cavitand in a sensor, according to certain embodiments of the disclosure, can generate a signal and/or signal pattern associated with the detection and/or identification of a particular substance associated with a specific health condition or disease. In some instances, a sensor including at least one supramolecule or cavitand can be tuned to generate a signal and/or signal pattern associated with the detection and/or identification of a predefined threshold, concentration and/or amount of a particular substance associated with a specific health condition or disease. In any instance, the use and implementation of a sensor with at least one supramolecule or cavitand in a sensor, according to certain embodiments of the disclosure, can provide selective and targeted ultra-high sensitivity to one or more substances.

In at least one embodiment, a sensor module, such as 108, can include a sensor with one or more flexible substrates with enhanced graphene blended with metal oxide nanoparticles. Each of the flexible substrates can include a thin-film deposition of graphene particles dispersed in an acrylic polymer. While intrinsic graphene is generally found to be highly sensitive in detecting certain gases such as CO, NOₓ, and NH₃, the relative selectivity can be greatly improved by using hybrid structures of graphene blended with metal oxide nanoparticles. For example, customized metal oxide nanoparticles can be blended with hybrid structures of graphene to enhance the composite sensor array sensitivity and selectivity for breath analysis of one or more VOCs. The electrochemical potential window of about 2.5 V for the graphene electrode in about 0.1 M phosphate-buffered saline with a pH value of about 7.0 can be comparable to the electrochemical potentials obtained for glassy carbon (GC) and boron-doped diamond. Further, alternating current (AC) impedance measured graphene charge-transfer resistance can be relatively smaller than other conventional electrodes. The electrochemical properties of enhanced graphene in such sensors can be operable to detect and identify the presence of certain VOCs with a relatively high degree of reliability and sensitivity. Thus, the use and implementation of one or more flexible substrates with enhanced graphene blended with metal oxide nanoparticles in a sensor, according to certain embodiments of the disclosure, can generate a signal and/or signal pattern associated with the detection and/or identification of a particular substance associated with a specific health condition or disease. In some instances, a sensor including one or more flexible substrates with enhanced graphene blended with metal oxide nanoparticles can be tuned to generate a signal and/or signal pattern associated with the detection and/or identification of a predefined threshold, concentration and/or amount of a particular substance associated with a specific health condition or disease. In any instance, the use and implementation of a sensor with one or more flexible substrates with enhanced graphene blended with metal oxide nanoparticles, according to certain embodiments of the disclosure, can provide selective and targeted ultra-high sensitivity to one or more substances.

In at least one embodiment, a sensor module, such as 108, can include at least one sensor with enhanced graphene blended with metal oxide nanoparticles having at least one supramolecule or cavitand used as a sensing material, wherein the supramolecule or cavitand is applied as a thin film deposition to the enhanced graphene. According to certain embodiments of the disclosure, a sensor module, such as 108, with at least one sensor with enhanced graphene blended with metal oxide nanoparticles having at least one supramolecule or cavitand used as a sensing material can generate a signal and/or signal pattern associated with the detection and/or identification of a particular substance associated with a specific health condition or disease. In some instances, a sensor including enhanced graphene blended with metal oxide nanoparticles having at least one supramolecule or cavitand used as a sensing material can be tuned to generate a signal and/or signal pattern associated with the detection and/or identification of a predefined threshold, concentration and/or amount of a particular substance associated with a specific health condition or disease. In any instance, the use and implementation of a sensor with enhanced graphene blended with metal oxide nanoparticles having at least one supramolecule or cavitand used as a sensing material, according to certain embodiments of the disclosure, can provide selective and targeted ultra-high sensitivity to one or more substances.

In at least one embodiment, a sensor module, such as 108, can include some or all of the following features including, but not limited to, relative resistance to or immunity to water vapor due to the hydrophobic nature of an associated membrane; no catalytic poisoning of the associated membrane, as is commonly observed for certain materials, such as doped SnO₂; no observed accumulative effects, which can be responsible for baseline drift in some sensors; and no porous metal layer, the adhesion of which may be prone to degradation, needed as an upper layer to the associated membrane.

In some cases, it can be useful to monitor the composition of inhaled gases, for example when administering gases to the subject such as anesthetics, nitric oxide, medications, and other treatments, monitoring pollutants or environmental effects, for a person respiring with the assistance of a ventilator, or for persons using breathing apparatus. Thus, in at least one embodiment, a sensor module, such as 108, can include at least one sensor with at least one substance operable to generate an electronic signal when detecting anesthetics, nitric oxide, medications, other treatments, pollutants, or environmental effects.

In at least one embodiment, a sensor module, such as 108, can include multiple sensors and/or sensor components organized into an array of sensors or a sensor array. The sensor array may include any number of particular sensors tuned to detect one or more specific substances, such as VOCs. Further, the sensor array may include any number of particular sensors tuned to increase the relative selectivity and sensitivity of the sensor array to detect and/or identify a specific amount or concentration of one or more substances, such as VOCs. For example, one sensor in a sensor array may be tuned to detect a first VOC, which may be associated with a first type of cancer, while another sensor in the same sensor array may be tuned to detect a second VOC, which may be associated with the first type of cancer and a second type of cancer, and yet another sensor in the same sensor array may be tuned to detect a third VOC, which may be associated with a third type of cancer, and so on. In another example, one sensor in a sensor array may be tuned to detect a first non-VOC, which may be associated with a first type of cancer, while another sensor in the same sensor array may be tuned to detect a first VOC, which may be associated with the first type of cancer and a second type of cancer, and yet another sensor in the same sensor array may be tuned to detect a chemical, which may be associated with the second type of cancer and a particular health condition such as diabetes, and so on.

In at least one embodiment, a sensor module, such as 108, can include multiple composite sensor arrays, wherein each array can include a set of one or more sensors. Each of the one or more sensors can be selected from a group comprising: an interdigitated electrode, a modified graphene electrode; a gold nanowire electrode; and an enhanced 2D metal carbide. The one or more selected sensors can be configured in an array operable to detect and/or identify a particular substance associated with a specific health condition or disease. Each sensor and/or array can be tuned to generate a signal and/or signal pattern associated with the detection and/or identification of a particular substance associated with a specific health condition or disease. In some instances, each sensor and/or array can be tuned to generate a signal and/or signal pattern associated with the detection and/or identification of a predefined threshold and/or range, concentration and/or amount of a particular substance associated with a specific health condition or disease. Thus, each sensor and/or array can generate a respective signal and/or signal pattern depending on the particular substance being detected and/or identified, or a predefined threshold and/or range of concentration and/or amount of a particular substance being detected and/or identified. In any instance, the use and implementation of composite sensor arrays, according to certain embodiments of the disclosure, can provide selective and targeted ultra-high sensitivity to one or more substances.

In at least one embodiment, a sensor module, such as 108, can include any number analytical devices or techniques, such as a mass spectrometer, chromatography device, calorimeter, spirometry, or other instruments for performing gas and/or flow analysis. Such analytical devices or techniques can further analyze the exhaled breath for detecting and/or identifying one or more substances. Example analytical devices or techniques can include, but are not limited to, gas chromatography, flame and/or combustion reactions detected using characteristic optical emission and/or absorption lines, hydrogen flame ionization, an indirect calorimeter, chemical detection methods, and colorimetry.

In at least one embodiment, a sensor module, such as 108, can include a flow rate sensor. The flow rate sensor can be operable to measure a flow rate of the exhaled breath within the housing 104 and/or mouth piece 106. For example, a sensor module, such as 108, can include any number of flow rate sensors, such as gas flow rate sensors, so as have the capabilities of a spirometer. The combination of gas flow rate measurement functionality and resulting capabilities may be useful for detecting respiratory components such as a nitric oxide diagnostic of asthma and other respiratory tract inflammations. The combination of respiratory component analysis and flow rate analysis can be helpful in diagnosing respiration disorders.

Turning back to FIG. 1, after the sensor module 108 has detected and identified one or more substances, the communication module 110 can transmit collected data, including information about the one or more substances from the sensor module 108 to a mobile communication device 112 and/or one or more remote server devices, such as 114 for processing by a respective biomarker processing module or engine, such as 122 and 140, which is described in more detail below.

For example, collected data may be transferred from the communication module 110, by way of direct attachment of the housing 104, to the mobile communication device 112. By way of example, a mobile communication device can be a smart phone, a portable computer or laptop computer, or a wireless phone. Collected data may also be transmitted by the communication module 110, via one or more networks 116, to a portable computer or laptop computer, an interactive television component (e.g. set-top box, web-TV box, cable box, satellite box, etc.), a desktop computer, a wireless phone, a wearable computing device, a smart watch, a wearable activity tracker, a remote server, or to a cloud-based computing or storage device. Any communication technique and/or protocol may be used, including wired and/or wireless communications, such as Bluetooth protocol, radio communication, IR communication, the Internet, a cellular connection, transferable memory sticks, wires, or other electromagnetic/electrical methods.

In at least one embodiment, transmission of the collected data from the sensor module 108 to a mobile communication device 112 and/or one or more remote server devices, such as 114, and subsequent storage by the mobile communication device 112 and/or one or more remote server devices, such as 114, can include any number of encryption techniques to facilitate compliance with any number of local, state, federal, or national laws or regulations, such as HIPAA (the Health Insurance Portability and Accountability Act), concerning personal information, medical or health information, and/or individual privacy rights. For example, collected data may be encrypted by mobile communication device 112 with a data or time stamp, and a user or patient identifier, and the encrypted collected data can be securely transmitted to one or more remote server devices, such as 114, for storage and/or processing by a respective biomarker processing module or engine, such as 122 and 140.

The example mobile communication device 112, shown in FIG. 1, can include one or more processors 118; a memory device 120 including the biomarker processing module 122 or engine, a diagnostic module 124, and an operating system (O/S) 126; one or more activity sensors 128; a network and input/output (I/O) interface 130; and an output display 132.

The example remote server computers 114, shown in FIG. 1, can include one or more processors 136; a memory device 138 including a biomarker processing module 140 or engine, a diagnostic module 142, an operating system (O/S) 144, and a database management system (DMBS) 146; a network and input/output (I/O) interface 148; and an output display 150. Each of the remote server computers 114 may include any number of processor-driven devices, including, but not limited to, a server computer, a personal computer, one or more networked computing devices, an application-specific circuit, a minicomputer, a microcontroller, and/or any other processor-based device and/or combination of devices.

Each of one or more processors 118, 136 may be implemented as appropriate in hardware, software, firmware, or combinations thereof. Software or firmware implementations of the one or more processors 118, 136 may include computer-readable or machine-readable instructions written in any suitable programming language to perform the various functions described.

Each memory device 120, 138 can be any computer-readable medium, such as a non-transitory medium, respectively coupled to the one or more processors 118, 136, such as random access memory (“RAM”), read-only memory (“ROM”), and/or removable storage devices. Each memory device 120, 138 may store one or more program modules, such as an operating system (OS) 126. The OS 126 may be any suitable module that facilitates the general operation of the remote server device 114, as well as the execution of other program modules. The one or more program modules may include a biomarker processing module 122, 140 or engine, and a diagnostic module 124, 142. The one or more program modules, such as the biomarker processing module 122, 140 or engine, and diagnostic module 124, 142, may include one or more suitable software modules and/or application programs. With respect to the remote server computer 114, the memory 138 can also store a database management system (DMBS) 146, which may be operable to generate and store any number of files and databases in any number of internal and/or external locations, such as in one or more datastores 134. Each of the program modules and the database management system (DMBS) 146 can include one or more computer-executable instructions operable to be read and executed by the one or more processors 118, 136.

### Example Biomarker Processing Module, Engine, And Methods

In the example shown in FIG. 1, a biomarker processing module, such as 122, can be operable to receive collected data from the sensor module 108 via the communication module 110 of the device 102, and can be further operable process the collected data associated with detection and identification of the one or more substances from the exhaled breath of a subject. The biomarker processing module 122, in some instances, may communicate, via the one or more processors 118 and/or network and I/O interface 130, with the communication module 110 to receive the collected data from the sensor module 108. Generally, the collected data can include one or more signals and/or signal patterns from the sensor module 108, wherein each of the respective signals and/or signal patterns may correspond to a particular sensor in the sensor module 108. Each signal may correspond with a different substance in an exhaled breath of a subject, and further correspond with a concentration and/or amount of the detected and identified substance. Further, the sensor module 108 may generate, for example, a signal pattern, which may reflect one or more substances, concentrations, and/or amounts of the substances detected and identified in the exhaled breath of a subject.

In any instance, the biomarker processing module 122 shown in FIG. 1 can determine, based at least in part on the collected data from the sensor module 108 via the communication module 110 of the device 102, a correlation of a detected and identified substance with a specific amount and/or concentration of the substance. For example, a signal or signal pattern received from the sensor module 108 can be correlated the biomarker processing module 122 with a concentration or amount of a specific substance identified in an exhaled breath of a subject. The correlated concentration or amount of the specific substance can be compared by the biomarker processing module 122 with one or more predefined signals and/or signal patterns corresponding to previously detected and identified substances stored in an associated memory, such as 120, or accessible by the mobile communication device 112 in, for example, one or more remote servers 114, associated memory 138, or other remote data storage devices or datastores, such as 134.

At least one difference from standard analytic techniques, a sensor module, such as 108, operating in conjunction with a biomarker processing module, such as 122, according to certain embodiments of the disclosure, can mimic mammalian olfaction in that it may not need to distinguish specific VOCs but is rather based at least in part on pattern recognition. In at least one embodiment, a sensor module, such as 108, can be used to generate one or more signals and/or signal patterns stored in a remote data storage device or datastore, such as 134, for subsequent analysis during a pattern recognition process, such as a neural network or pattern recognition algorithm. Further, the sensor module 108 and biomarker processing module 122 can be used to classify any number of unknown exhaled breath samples from various subjects. One skilled in the art will recognize that a suitable pattern recognition process can be established by varying the size of a training set of signals and/or signal patterns for a pattern recognition or matching process as well as determining how well the training set of signals and/or signal patterns represent signals and/or signal patterns in previously tested populations of subjects. In at least one embodiment, a sensor module, such as 108, and biomarker processing module, such as 122, can be improved by implementing both with a combination of other techniques and/or other sensors, such that specific VOCs can be identified by one sensor, for example, a gas chromatography-mass spectrometry (GC-MS) sensor, which may be used to select one or more other sensors most sensitive to certain target compounds. In this manner, in certain embodiments, for a sensor module, such as 108, the same sensors may be used in some or all of the sensor arrays, and each sensor may be individually tuned to be cross-sensitive to various VOCs. This can result in the same sensor module, such as 108, generating a unique signal and/or signal pattern, that is, a digital biomarker, for each disease examined using the same sensors in the same sensor arrays of the sensor module 108.

It is known in the art that certain micro-organisms can produce patterns of VOCs that can be affected by the type and age of the culture. These VOC patterns can be used as biomarkers for detecting food spoilage as well as biomarkers for certain diseases. Some examples of conventional electronic gas sensing devices for the detection of VOCs that are applicable to the food industry include U.S. Pat. Nos. 4,399,687; 6,170,318; 6,234,006; 6,428,748; 6,537,802; 6,658,915; 6,767,732; and 6,837,095; and U.S. Patent Application Nos. 2006/0078658 and 2006/0191319, the contents of these prior references are incorporated herein by reference.

Other conventional odor detecting devices and systems employing sensor arrays used in certain medical applications are known in the art. Some examples are disclosed in U.S. Pat. Nos. 6,173,602; 6,319,724; 6,411,905; 6,467,333; 6,540,691; 6,609,068; 6,620,109; 6,703,241; 6,839,636; 6,841,391; 7,052,854; 7,153,272; 7,241,989; and in U.S. Patent Application Nos. 2001/0041366; 2002/012762; 2005/0037374; 2005/0065446; 2006/0151687; 2006/0160134; 2006/0277974; and 2007/0062255, the contents of these prior references are incorporated herein by reference. However, the use of various analysis instruments in many of these conventional devices and systems can be relatively complex, time consuming, and the sensitivity may be limited to parts per million (ppm) rather than parts per billion (ppb), sometimes needed for detecting certain biomarkers for various health conditions and diseases.

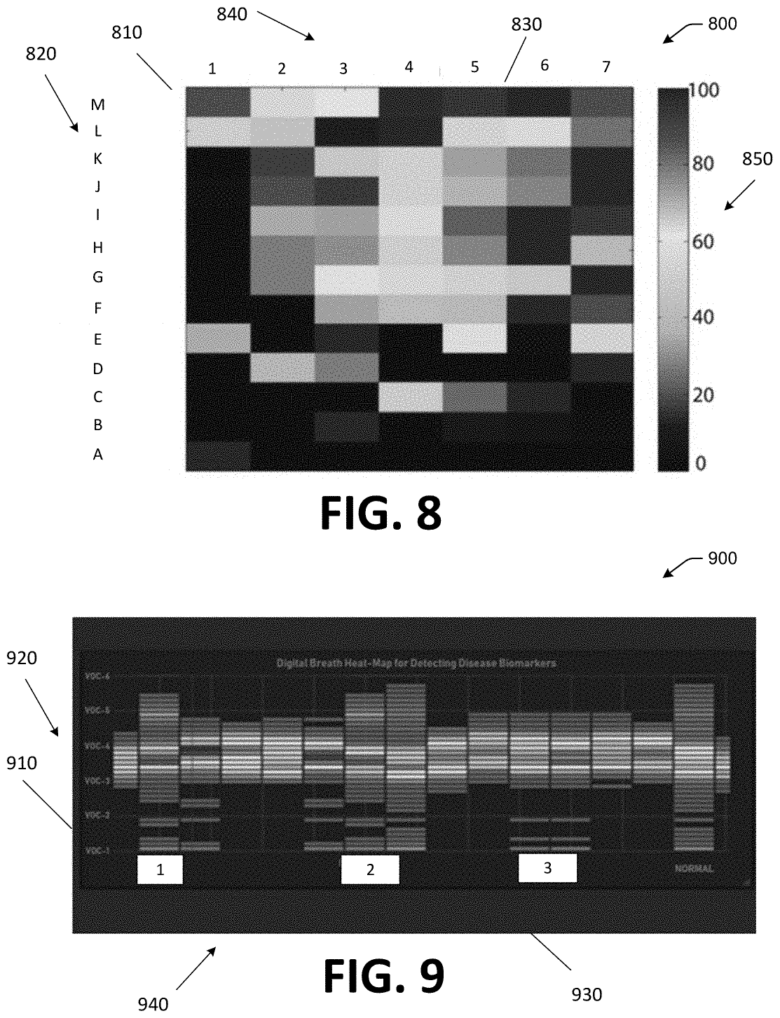

In at least one embodiment, a biomarker processing module, such as 122, can be operable to process the collected data from a sensor module, such as 108, via a neural network or pattern recognition algorithm, wherein a result from the biomarker processing module 122 can be output to a display 132 associated with the mobile communication device 112. For example, when data is processed by a neural network or pattern matching algorithm, an initial result may include identification of one or more substances in the exhaled breath, identification of a unique sensor derived signal and/or signal pattern of the one or more substances in the exhaled breath, an identification of and/or correlation with a health condition or disease, and/or an identification of and/or correlation with any number of substances in a subject's exhaled breath. The biomarker processing module 122 can, in certain instances, communicate with a diagnostic module, such as 124, to prepare and present a suitable output or result for transmission to the display 132 associated with the mobile communication device 112. Example outputs for a display 132 can include, but are not limited to, a heat map, a graph, a chart, a concentration of a specific substance, an amount of a specific substance, or an indication of the presence of a specific substance. FIGS. 8 and 9, described in more detail below, depict example outputs from a biomarker processing module, such as 122, or engine, or from a diagnostic module, such as 124, for a display 132.

An example neural network can be initially created and/or subsequently trained by receiving and storing any number of previously detected and identified signals and/or signal patterns of one or more substances from the exhaled breaths of any number of subjects. In some embodiments, the signals and/or signal patterns of multiple substances can be received and stored, wherein each signal and/or signal pattern may be correlated with one or more health conditions or diseases. Further, in some embodiments, a signal and/or signal pattern may be associated with the presence of a respective substance. Furthermore, in some embodiments, each signal and/or signal pattern may be associated with concentration and/or amount of a specific substance. In some embodiments, a signal and/or signal pattern may be associated with the presence of a specific combination of multiple substances. In certain embodiments, a signal and/or signal pattern may be associated respective concentrations and/or amounts of multiple substances in combination with each other. In any instance, the signals and/or signal patterns of any number of substances can be received and stored, wherein each signal and/or signal pattern may be correlated with one or more health conditions or diseases.

An example pattern recognition algorithm can be an algorithm operable to seek a best match and/or relatively high confidence score in matching a signal and/or signal pattern to one or more previously stored signals and/or signal patterns correlated with one or more health conditions or diseases.

In addition to a neural network and pattern recognition algorithm, any number of mathematical and computational tools and techniques can be implemented by a biomarker processing module, such as 122 or 140, to process collected data from a sensor module, such as 108, including, but not limited to, using feature extraction and feature selection processes in conjunction with an artificial neural network (ANN), artificial intelligence techniques, a multifactorial approach, leave-one-out cross-validation (LOOCV), nonlinear support vector machine (SVM), multi-layer perception (MLP), generalized regression neural network (GRNN), fuzzy inference systems (FIS), self organizing map (SOM), radial bias function (RBF), genetic algorithms (GAS), neurofuzzy systems (NFS), adaptive resonance theory (ART) and statistical methods such as canonical discriminant analysis, canonical correlation, principal component analysis (PCA), partial least squares (PLS), multiple linear regression (MLR), principal component regression (PCR), discriminant function analysis (DFA) including linear discriminant analysis (LDA), and cluster analysis including nearest neighbor.

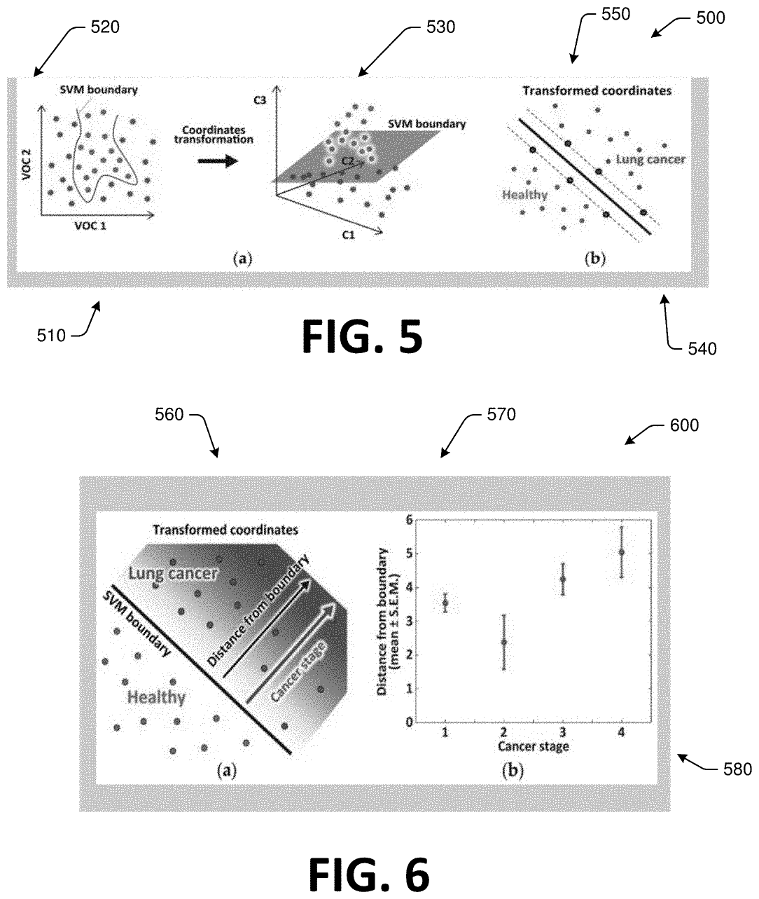

In at least one embodiment, as depicted in FIGS. 5 and 6, a nonlinear support vector machine (SVM) analysis 500 can be applied by a biomarker processing module, such as 122, to collected data from exhaled breath of a subject. In this example, a two-class data set can include data from at least two VOCs, such as VOC1 510 and VOC2 520. The data sets can be transformed into a different coordinate space 530 where the datasets can be classified or otherwise separated by a relatively flat boundary, called a SVM boundary 540 or classification boundary. The boundary between the two data sets can be determined using data points called support vectors 550. The number of support vectors 550 can be relatively small to avoid overfitting the data points. In this example, a diagnosis of a particular health condition or disease stage can correlate with a relative distance 560 from the SVM boundary 550 in the transformed coordinates space. In another view of the data illustrating the relative distance of certain from the boundary, the y-axis 570 can indicate the distance from the SVM boundary, such as in mean +/−S.E.M., and the horizontal axis 580 can indicate a health condition or disease stage, such as stage 1 to 4. Generally, a correlation can be made between a health condition or disease stage and distance from the SVM boundary. Data points relatively near the SVM boundary can contain a property of each class because SVM can provide a boundary between the two-class data points. In other words, the data points that are relatively far from the SVM boundary usually have the specific property of their respective class. In the SVM diagnosis, the data samples of relatively low health condition or disease stages are generally located near the SVM boundary and those of relatively high stages are far from the SVM boundary. One skilled in the art will recognize that distance-based feature extraction has been theoretically studied and applied to MRI images of the brain for other types of health condition or disease diagnosis. Thus, in the context of the above example for analyzing VOCs in an exhaled breath of a subject, the computed distances of the health condition or disease samples from the SVM boundary can be performed using the best VOC combination in the true positive rate (TPR) rank with the leave one out (LOO) fashion. Further, test sample distances from the SVM boundary can be computed by the biomarker processing module, such as 122, developed from any remaining learning samples. The relatively higher health condition or disease stage samples can be located relatively far from the SVM boundary. This illustrates that, in certain instances, the SVM diagnosis could be used for estimating the health condition or disease stage of a patient. Thus, first-stage patients may have relatively long distances from the SVM boundary.

In at least one embodiment, a leave-one-out cross-validation (LOOCV) analysis can be used to evaluate the SVM diagnosis for a given data set, such as in the previous example. Leave-one-out cross-validation (LOOCV) has been previously used in biology and breath gas analysis. In the LOOCV analysis of this example, one data point can be left out of the data set to evaluate the accuracy of the SVM diagnosis, while the remaining data points can be used to train a classifier. Then, the left-out data point can be diagnosed by the trained classifier. This analysis can be repeated for each sample to compute a true positive rate (TPR=TP/(TP+FN)), true negative rate (TNR=TN/TN+FP), and accuracy (ACC=(TP+TN)/(TP+FN+TN+FP)). In one instance, 29 healthy controls can be used as true negative samples. These values can equal 100% if a completely accurate diagnosis is achieved. LOOCV analysis can be applied to some or all of the received VOC combinations to screen the relative effective combinations for health condition and/or disease diagnosis.

Turning back to FIG. 1, in at least one embodiment, a biomarker processing module, such as 122, can be operable to communicate via one or more networks 116 with one or more remote server computers 114 to transmit some or all of the collected data from the device 102 to the one or more remote server computers 114 for processing and/or analysis. In some instances, a biomarker processing module, such as 122, can be operable to communicate via one or more networks 116 with one or more remote server computers 114 to transmit processed data from the biomarker processing module 122 to the one or more remote server computers 114 for additional processing and/or analysis.

In at least one embodiment, a biomarker processing module, such as 140, can be further operable to process the collected data in conjunction with other sensor data, such as from the one or more activity sensors 128 and/or wearable computing devices 128N. The one or more activity sensors 128 can include, but are not limited to, a respiratory analyzer device, a metabolic rate meter, a pedometer, an accelerometer, an activity tracker or meter, a heart rate or pulse meter, a blood pressure device, or any type of sensor that can be incorporated into or operate with a mobile communication device 112. In some embodiments, a wearable computing device, such as 128N, may be separate from and external to the mobile communication device 112, such as a fitness or personal activity tracker. In any instance, measurements from these separate and external activity sensors can be transmitted to the mobile communication device 112 and/or biomarker processing module 122 via any communication technique and/or protocol, including wired and/or wireless communications, such as Bluetooth protocol, radio communication, IR communication, the Internet, a cellular connection, transferable memory sticks, wires, or other electromagnetic/electrical methods.

In at least one embodiment, one or more sensors can include a metabolic rate meter operable for providing a metabolic rate for a subject. For example, a metabolic rate meter can include a nanoparticle flow rate sensor, a microelectronic flow rate sensor, a nanoparticle oxygen sensor, a microelectronic oxygen sensor, a nanoparticle carbon dioxide sensor, a microelectronic carbon dioxide sensor, and/or any number of ultrasonic transducers. For instance, a pair of ultrasonic transducers, nanoparticle flow sensors, or microelectronic flow sensors can be measure the density of exhaled air to determine oxygen and carbon dioxide concentrations in the exhaled air, which can be useful to analyze the metabolic rate for a subject.

In the example shown in FIG. 1, a diagnostic module, such as 124, can be operable to receive processed data from the biomarker processing module 122, and can be further operable to determine, based at least in part on the processed data from the biomarker processing module 122, one or more diagnoses and/or treatments for a subject. The diagnostic module 124, in some instances, via the one or more processors 118 and/or network and I/O interface 130, may communicate with the biomarker processing module 122 of the device 102 to receive processed data. In such instances, the diagnostic module 124 can be further operable to determine, based at least in part on the processed data from the biomarker processing module 122, one or more diagnoses and/or treatments for a subject.

In many instances, certain treatments for health conditions and/or diseases can include any number of conventional treatments. For example, a treatment to address cancer can include, but is not limited to, surgical removal, radiation or radiotherapy, chemotherapy, hormone therapy, targeted cancer drugs, immunotherapy, stem cell and bone marrow transplants, gene therapy, and personalized medicine. One skilled in the art will recognize that any number of conventional treatments can be implemented to address a particular health condition or disease, and one skilled in the art will recognize how a diagnostic module, such as 124, can be implemented to facilitate treatment to address a particular health condition or disease. For example, various actions by a diagnostic module, such as 124, to facilitate treatment to address cancer can include, but are not limited to, scheduling resources, time, and personnel to perform and/or administer any of a surgical removal, radiation or radiotherapy, chemotherapy, hormone therapy, targeted cancer drugs, immunotherapy, stem cell and bone marrow transplants, gene therapy, and/or personalized medicine.

In other embodiments, a diagnostic module, such as 124, can be operable to receive processed data from the biomarker processing module 122, and can be further operable to determine, based at least in part on the processed data from the biomarker processing module 122, one or more metabolic and/or substance indicators for a subject. Suitable indicators can include, but are not limited to, numeric data, graphical data, colored indicia, audio data, visual data, haptic data, and any combination thereof. The diagnostic module 120, in some instances, via the one or more processors 118 and/or network and I/O interface 130, may communicate with the biomarker processing module 122 to receive the processed data. The processed data may include a ketone signal based on the exhaled breath, and a metabolic rate determined from physical activity measured by the one or more activity sensors 128. In such instances, the diagnostic module 120 can be further operable to determine, based at least in part on the processed data from the biomarker processing module 122, one or more metabolic and/or substance indicators for a subject.

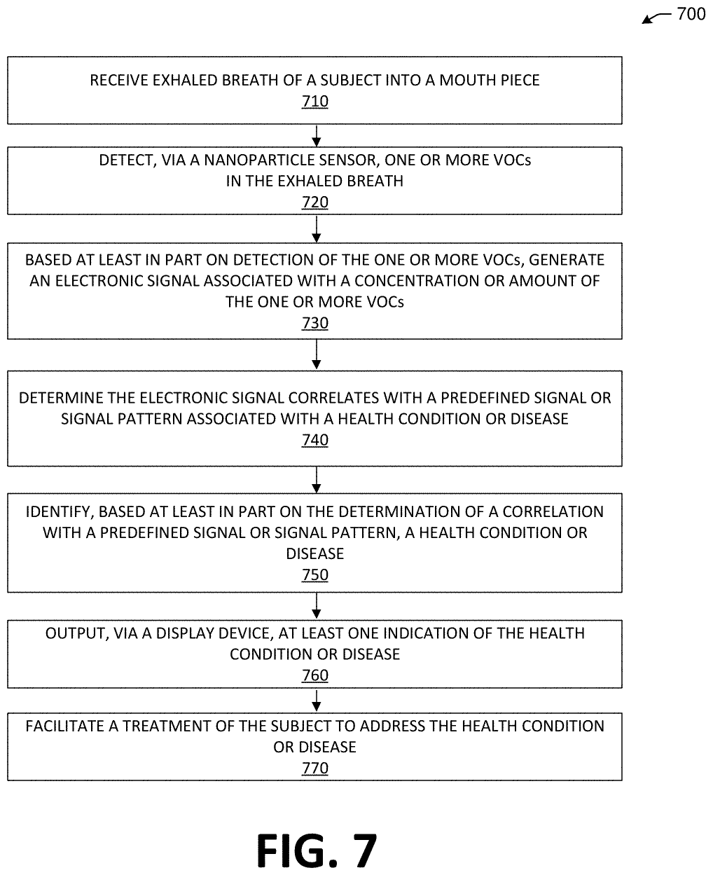

FIG. 7 depicts an example method 700 for detecting certain substances including VOCs in an exhaled breath of a subject, according to one example embodiment of the disclosure. The example method 700 can further be used to diagnose and treat a health condition or disease in a subject, according to one example embodiment of the disclosure. The method 700 can be implemented with the system 100 and device 102 shown and described in FIG. 1, and may also be implemented with one or more of the example sensors 200, 300, 400 in FIGS. 2, 3, and 4. One skilled in the art will recognize that the method 700 can be implemented with respect to any number of other substances including VGs, ketones, etc.

In FIG. 7, the method 700 begins at block 710 with receiving an exhaled breath of a subject into a mouth piece. With respect to FIG. 1, a subject can breathe into the mouth piece 106 of the device 102, wherein the exhaled breath of the subject can be received into the mouth piece 106. In block 720, one or more VOCs in the exhaled breath can be detected via a nanoparticle sensor. With respect to FIG. 1, the exhaled breath from the mouth piece 106 can pass over, through, and/or adjacent to the sensor module 108, wherein the one or more VOCs in the exhaled breath can be detected via a nanoparticle sensor. In block 730, based at least in part on the detection of the one or more VOCs, an electronic signal associated with a concentration or amount of the one or more VOCs can be generated. With respect to FIG. 1, the sensor module 108 can detect one or more VOCs in the exhaled breath of the subject, and the sensor module 108 can generate an electronic signal that corresponds with a concentration or amount of the one or more VOCs in the exhaled breath of the subject. In block 740, the electronic signal can be determined to correlate with a predefined signal or signal pattern associated with a health condition or disease. With respect to FIG. 1, the electronic signal can be communicated by the communication module 110 of the device to a biomarker processing module 122 of the mobile communication device 112, and the electronic signal can be determined by the biomarker processing module 122 to correlate with a predefined signal or signal pattern associated with a health condition or disease. In block 750, the determination that the electronic signal from the sensor module 108 correlates with a predefined signal or signal pattern associated with a health condition or disease, can be used to identify a health condition or disease. Turning to FIG. 1, the determination by the biomarker processing module 122 that the electronic signal from the sensor module 108 correlates with a predefined signal or signal pattern associated with a health condition or disease, can be used by the biomarker processing module 122 and/or the diagnostic module 124 to identify the health condition or disease. In block 760, at least one indication of the health condition or disease can be output via a display device. Turning to FIG. 1, the biomarker processing module 122 and/or the diagnostic module 124 can generate and output via the display 132 of the mobile communication device 112, an indication of the health condition or disease. In block 770, a treatment of the subject can be facilitated to address the health condition or disease. Turning to FIG. 1, the diagnostic module 124 can facilitate treatment of the subject to address the health condition or disease.

One skilled in the art will recognize that the various components for the above example system 100 and device 102 of FIG. 1, example sensors 200, 300, 400 in FIGS. 2, 3, and 4 and example methods 700 in FIG. 7 can be, in accordance with embodiments of the disclosure, assembled and organized in other operating architectures than those described in these embodiments. One skilled in the art will recognize that various sensors and sensor modules can be implemented with the system 100 and device 102 of FIG. 1, and certain operations of the method 700 of FIG. 7, to detect, identify, classify, and quantify any number and any type of substance, such as volatile organic compounds (VOCs), volatile gases (VGs), and ketones, in an exhaled breath of a subject or person in real-time. One skilled in the art will recognize that various techniques, tools, and algorithms can be implemented with the biomarker processing module 122 to detect, identify, classify, and quantify any number and any type of substance, such as volatile organic compounds (VOCs), volatile gases (VGs), and ketones, in an exhaled breath of a subject or person in real-time. One skilled in the art will recognize that various techniques, tools, and algorithms can be implemented with the biomarker processing module 122 and/or diagnostic module 124 to detect and identify a health condition or disease in a subject or person in real-time. One skilled in the art will recognize that various techniques, tools, and methods can be implemented with the diagnostic module 124 to facilitate treatment or otherwise address a health condition or disease in a subject or person.

### Example Health, Exercise, and Diet Monitoring Systems, Devices, and Methods

Certain embodiments described above can be used in various health, exercise, and diet monitoring systems, methods, and devices. The system 100 and/or device 102 shown in FIG. 1 can be adapted with a respiratory analyzer device to be a field measurement system operable to detect and classify exhaled gases within a breath sample of a user and/or subject who is on a diet or weight control program or who is diabetic. In at least one embodiment, the system 100 and/or device 102 of FIG. 1 can include a respiratory analyzer device operable to detect and identify one or more VOCs, such as VGs and/or ketones, in an exhaled breath of a subject. The resulting VOC, VG, and ketone measurements from the respiratory analyzer device, system 100, and/or device 102 can be used in an improved weight loss program involving an exercise component. The respiratory analyzer device may be an electronic exhaled gas sensing apparatus operable to detect exhaled gases and other substances, which may be ketones such as acetone. The respiratory analyzer device may be used for real-time site assessment and monitoring activities associated with diet and weight loss as well as monitoring and detection of ketones in a subject with diabetes.

Various conventional technologies and techniques have previously been used to detect certain substances, including U.S. Pat. No. 4,114,422 to Hutson (acetone), and U.S. Pat. No. 4,758,521 to Kundu (ketones). However, none of these conventional technologies and techniques address the needs that various embodiments of the disclosure address. Each of these prior references is hereby incorporated by reference.

Further, conventional technologies and techniques have previously been used to estimate resting metabolic rate using the Harris-Benedict equation, as discussed by U.S. Pat. No. 5,839,901 to Karkanen, as well as measuring metabolic rate using various gas sampling techniques and differential pressure based flow rate sensors, as discussed by U.S. Pat. No. 5,705,735 to Acorn. However, none of these conventional technologies and techniques address the needs that various embodiments of the disclosure address. Each of these prior references is hereby incorporated by reference.

By way of further example of at least one embodiment of the disclosure, when a subject is using a respiratory analyzer device incorporated with the system 100 and/or device 102 of FIG. 1, one or more activity sensors (e.g. pedometer, accelerometer), such as 128, or separate from the mobile communication device 112 (e.g., personal activity tracker), such as 128N, can measure the subject's activity, such as walking, jogging, or running. A sensor module, such as 108 in FIG. 1, could be equipped with a nanoparticle sensor with additional ketone sensing capability to monitor the subject's oxygen intake rate and hence metabolic rate, and also detect the attainment of a certain acetone level in the subject's exhaled breath, indicating, in some instances, the onset of fat catabolism. The collected data from the sensor module 108 and one or more activity sensors 128, 128N can be transmitted by a communication module, such as 110, to a subject's mobile communication device, such as 112, or smart phone, and further transmitted to a remote server device, such as 114, via a network, such as 116, including the Internet or cloud. In any instance, the collected data can be used by a biomarker processing module, such as 122, 140, and/or diagnostic module, such as 124, 142, to create a model of the subject's physiological response to exercise.

An example model could include various historical activity data of the subject, and can include any number of indications relative to a specific type of activity, such as walking, jogging, or running, and further indications of the subject's physiological response to the type of activity. For example, the subject's oxygen intake rate and hence metabolic rate could be indicated, and changes over time relative to the subject's particular type of physical activity could be indicated, thus illustrating the subject's physiological response to exercise.

During a daily exercise routine, a signal from the one or more activity sensors 128, 128N can be transferred to the subject's mobile communication device 112 or smart phone, and further transmitted to a remote server device 114 via a network 116, such as the Internet or cloud. The subject's mobile communication device 112 or smart phone can then be used to provide quantitative feedback to the subject regarding the benefits of the exercise. For example, the mobile communication device 112 or smart phone may be used to indicate feedback, such as the calories burned, the time the exercise must continue for the onset of fat burning, and/or an estimate of fat grams burned. This level of feedback to the subject may be an improvement over previous weight control and exercise programs, and may also be a motivational factor for the subject to continue with the exercise.

By way of another example, a diet and exercise control program, device, and method can be implemented for a subject suffering from diabetes. The subject could carry a mobile communication device 112 or smart phone, and may also have a glucose sensor transmitting blood glucose levels to the mobile communication device 112 or smart phone using a wireless transmission protocol such as Bluetooth. Dietary intake data can be entered by the subject into the mobile communication device 112 or smart phone. The smart phone can be used to track dietary intake and blood sugar levels, estimate possible future deviations of blood sugar from an acceptable range, and provide warnings and advice to the subject. Indirect calorimetry, for instance, can be used to determine the metabolic rate of the subject. An activity sensor, such as 128, 128N, can be used to provide a signal correlated with the subject's physical activity data. The physical activity data can be transmitted to the mobile communication device 112 or smart phone, preferably using Bluetooth. Breath ketone sensing can be used to detect the onset of the dangerous condition of ketoacidosis.

An indication or communication including a warning to a subject regarding the onset of ketoacidosis can be generated and transmitted to the subject's mobile communication device 112 or smart phone, which could include a smart phone application or app carried by the subject. The subject's mobile communication device 112 or smart phone could also include or otherwise communicate with a blood glucose sensor, and a respiratory analyzer (which could function as an indirect calorimeter and respired volatile organics detector). Collected data may also be transferred to a biomarker processing module, such as 122, 140, and/or diagnostic module, such as 124, 142, via the network 116, such as the Internet or cloud, for further analysis.

The following example relates to exercise management. A subject who is exercising can carry a portable ketone analyzer, which can be in communication with or incorporated into the system 100 and/or device 102 shown in FIG. I. The portable ketone analyzer can include a tube that is breathed through, and a nanoparticle ketone detector disposed on one wall of the tube. The portable ketone analyzer may be relatively small, such as the size of a human finger. The subject may periodically breathe or blow through the tube into the portable ketone analyzer, which can determine whether the subject is burning fat. In some instances, the portable ketone analyzer may prompt the subject to periodically breathe or blow through the tube into the portable ketone analyzer, or the portable ketone analyzer may indicate to the subject that analysis may be needed after a certain period of time has passed. Further, a separate exercise monitor may communicate with the portable ketone analyzer, and indicate that theb subject's breath should be analyzed after a certain set of predefined conditions are met. A biomarker processing module, such as 122, 140, and/or diagnostic module, such as 124, 142, may communicate certain results back the exercise monitor, may provide a confirmation of results such as by a chime or other indication indicating whether the subject is burning fat, or may store certain results versus time onto a data storage device, such as 120, 134, or 138. The collected data can be transmitted from the mobile communication device 112 or smart phone in real-time via the network 116, such as the Internet or cloud, for further analysis.

In at least one embodiment, a method for facilitating exercise in a subject can include some or all of the following actions or operations including, but not limited to, monitoring a metabolic rate of a subject during an exercise or physical activity; correlating the exercise or physical activity with metabolic rate; detecting the presence of one or more organic compounds in an exhaled breath of the subject, indicative of one or more fat metabolizing processes in the subject; determining an effect of exercise or physical activity on fat burning; providing feedback to the subject during future repetition of the exercise or physical activity, in terms of an effect of the exercise or physical activity on metabolic rate and fat burning, whereby the subject is encouraged to continue exercising or performing the physical activity by the provision of the feedback.

In at least one embodiment, an improved respiratory analyzer for a subject can include some or all of the following elements including, but not limited to, a flow path, through which the subject breathes; a metabolic rate meter, providing metabolic data correlated with the metabolic rate of the subject; a ketone sensor, providing a ketone signal correlated with a concentration of one or more respiratory components in an exhaled breath of the subject, wherein the one or more respiratory components are correlated with a level of ketone bodies in the blood of the subject; an output display or device; and an electronic circuit operable to receive the ketone signal and the metabolic data, and further operable to provide a visual or other indication of the metabolic rate and the ketone signal via the output display or device.

In at least one embodiment, a nanoparticle respiratory analyzer for a subject can include some or all of the following elements including, but not limited to, a flow path, through which the subject breathes; a sensor, providing an exhaled volatile gas signal correlated with a concentration of one or more respiratory components in an exhaled breath of the subject, wherein the concentration of the one or more respiratory components are correlated with a level of which are volatile organic compounds (VOCs) including volatile gases (VGs) in the blood of the subject; an output display or device; an electronic circuit operable to receive the VGs and the metabolic data, and further operable to provide a visual or other indication of the VG signal via the output display or device. In certain aspects, VG signal patterns can be standardized in collection and analysis and may be specifically correlated with certain correlated with corresponding disease states or exhaled anesthetic gases that can be identified using a nanoparticle VG sensor, which may be incorporated into or otherwise function with the nanoparticle respiratory analyzer.

FIGS. 8 and 9 illustrate example heat map graphics generated by a biomarker processing module or engine, and/or a diagnostic module, according to one example embodiment of the disclosure. In the heat map graphic 800 shown in FIG. 8, the vertical axis 810 illustrates a number of substances (A-M) 820, such as VOCs, VGs, or ketones, which were tested in an exhaled breath of one or more samples or subjects. The horizontal axis 830 illustrates a number of samples (1-7) 840, including, for example, at least one control and a number of uniquely identified samples. In any instance, the vertical heat bar 850 ranging from 0 to 100, illustrates a range of color values for which each of the substances 820 was detected and identified in the exhaled breaths of each of the tested samples 840. Thus, a respective shade of the color values corresponds with a concentration and/or amount of a substance detected and identified in the exhaled breath samples. For example, a relatively low concentration and/or amount can appear as a blue color corresponding to about 0 to 40, a relatively medium concentration and/or amount can appear as a green to light yellow color corresponding to about 40 to 60, a upper medium concentration and/or amount can appear as a dark yellow to light orange color corresponding to about 60 to 80, and a relatively high concentration and/or amount can appear as a dark orange color to red color corresponding to about 80 to 100. In this manner, an observer can readily evaluate data presented in the heat map graphic 800 to determine which substances have a relatively low and/or high concentration and/or amount for a particular subject or sample.

FIG. 9 depicts another example heat map graphic generated by a biomarker processing module or engine, according to one example embodiment of the disclosure. Similar to the graphic of FIG. 8, in this heat map graphic 900 shown in FIG. 9, the vertical axis 910 illustrates a number of substances 920, such as VOCs, VGs, or ketones, labeled here as VOC-1, VOC-2, VOC-3, VOC-4, VOC-5, and VOC-6, which were tested in an exhaled breath of one or more samples or subjects. The horizontal axis 930 illustrates a number of respective samples 940, labeled 1-3, which can be different health conditions, such as any number of different types of cancer, and a normal or control sample. In any instance, the range of color values for which each of the substances 920 was detected and identified in the exhaled breaths of each of the tested samples 940. Thus, a respective shade of the color values corresponds with a concentration and/or amount of a substance detected and identified in the exhaled breath samples. For example, a relatively low concentration and/or amount can appear as a blue color, a relatively medium concentration and/or amount can appear as a green to light yellow color, a upper medium concentration and/or amount can appear as a dark yellow to light orange color, and a relatively high concentration and/or amount can appear as a dark orange color to red color. In this manner, an observer can readily evaluate data presented in the heat map graphic 900 to determine which substances have a relatively low and/or high concentration and/or amount for a particular subject or sample.

One skilled in the art will recognize that a variety of user interfaces can be generated by a biomarker processing module or engine, and/or a diagnostic module for output to a display of a mobile communication device or other processor-based device. Such user interfaces can include, but are not limited to, graphics, sound, colors, text, haptics, graphs, charts, tables, and any combination thereof.

The disclosure is described above with reference to certain block and flow diagrams of systems, methods, apparatuses, and/or computer program products according to example embodiments of the disclosure. It will be understood that one or more blocks of the block diagrams and flow diagrams, and combinations of blocks in the block diagrams and the flow diagrams, respectively, can be implemented by computer-readable program instructions. Likewise, some blocks of the block diagrams and flow diagrams may not necessarily need to be performed in the order presented, or may not necessarily need to be performed at all, according to some embodiments of the disclosure. Various block and/or flow diagrams of systems, methods, apparatus, and/or computer program products according to example embodiments of the invention are described above. It will be understood that one or more blocks of the block diagrams and flow diagrams, and combinations of blocks in the block diagrams and flow diagrams, respectively, can be implemented by computer-readable program instructions. Likewise, some blocks of the block diagrams and flow diagrams may not necessarily need to be performed in the order presented, or may not necessarily need to be performed at all, according to some embodiments of the disclosure.

These computer-executable program instructions may be loaded onto a special purpose computer or other particular machine, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, embodiments of the disclosure may provide for a computer program product, comprising a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or operations to be performed on the computer or other programmable apparatus to produce a computer-implemented process such that the instructions that execute on the computer or other programmable apparatus provide elements or operations for implementing the functions specified in the flow diagram block or blocks.

Accordingly, blocks of the block diagrams and flow diagrams support combinations of means for performing the specified functions, combinations of elements or operations for performing the specified functions and program instruction means for performing the specified functions. It will also be understood that each block of the block diagrams and flow diagrams, and combinations of blocks in the block diagrams and flow diagrams, can be implemented by special purpose, hardware-based computer systems that perform the specified functions, elements or operations, or combinations of special purpose hardware and computer instructions.

Many modifications and other embodiments of the example descriptions set forth herein to which these descriptions pertain will come to mind having the benefit of the teachings presented in the foregoing descriptions and the associated drawings. Thus, it will be appreciated that the disclosure may be embodied in many forms and should not be limited to the example embodiments described above. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A system for detecting and identifying one or more volatile organic compounds (VOCs) in exhaled breath of a subject, the system comprising:
a mouth piece connected to a housing, the mouth piece operable to receive the exhaled breath of the subject;
a sensor module disposed in the housing, the sensor module operable to detect the one or more VOCs in the exhaled breath, and further operable to collect data associated with detection of the one or more one or more VOCs; and
a communication module disposed in the housing and in communication with the sensor module, the communication module operable to transmit collected data from the sensor module to a remote processor for analysis of the collected data.

2. The system of claim 1, wherein the sensor module comprises:
at least one sensor component operable to detect one or more VOCs, wherein the sensor component comprises at least one of the following: a MXene; a customized form 2D Mn+iXn (Ts) MXene composition; a 2D (two-dimensional) Ti3C2 nanosheet; Ti3C2Tx; Ti₃C₂(OH)₂; Ti₃C₂ MXene; a 3D MXene (3D-M) framework; one or more metal oxide nanoparticles blended with a hybrid structure of graphene; a conducting polymer such as polyaniline (PAni) and polypyrrole (PPY); a supramolecule; a cavitand (macrocyclic compounds based on resorcinarenes); or a relatively thin film of a supramolecule or a calixresorcinarene.

3. The system of claim 1, wherein the sensor module comprises:
at least one array of respective sensors, wherein each sensor is operable to detect at least one VOC.

4. The system of claim 1, wherein the VOCs comprise at least one of the following: a ketone, acetone, an aldehyde, an acetaldehyde, a hydrocarbon, an alkane, a pentane, an alkene, or a fatty acid.

5. The system of claim 1, wherein the one more VOCs is associated with a health condition comprising at least one of the following: diabetes, high or low blood glucose levels, ketoacidosis, lung cancer, breast cancer, a digestive cancer, gastric cancer, peptic ulcer, colorectal cancer, prostate cancer, head-and-neck cancer, stomach cancer, liver cancer, kidney disease, or neurodegenerative disease.

6. The system of claim 1, further comprising:
a biomarker processing module, in communication with the sensor module, and operable to process the collected data associated with detection of the one or more VOCs and to identify the one or more VOCs.

7. The system of claim 6, wherein the biomarker processing module is further operable to process the collected data via a neural network or pattern recognition algorithm, wherein a result from the biomarker processing module is received by the communication module for output to a mobile communication device associated with the subject.

8. The system of claim 6, wherein the biomarker processing module is further operable to process the collected data in conjunction with other sensor data, wherein a result from the biomarker processing module is received by the communication module for output to a mobile communication device associated with the subject.

9. The system of claim 1, further comprising:
an activity sensor operable to detect or measure one or more physical actions by the subject, and further operable to collect data associated with detection or measurement of the one or more physical actions; and
wherein the communication module is in communication with the activity sensor, and the communication module is further operable to transmit collected data from the activity sensor.

10. The system of claim 1, wherein the communication module is further operable to communicate with a mobile communication device associated with the subject, wherein the mobile communication device receives the collected data from the sensor module and the collected data from the activity sensor.

11. The system of claim 1, wherein the communication module is further operable to transmit collected data via at least one of the following: IR (infrared) communication, wireless communication, a Bluetooth protocol wireless communication, a direct wired connection, or to a remote memory storage device.

12. A method comprising:
receiving an exhaled breath of the subject;
detecting, via a nanoparticle sensor, one or more VOCs in the exhaled breath;
based at least in part on detection of the one or more VOCs, generating an electronic signal associated with a concentration or amount of the one or more VOCs; and
outputting, via a display device, an indication of a health condition or disease associated with the concentration or amount of the one or more VOCs.

13. The method of claim 12, further comprising:
determining the electronic signal correlates with a predefined signal or signal pattern associated with a health condition or disease; and
identifying, based at least in part on the determination of a correlation with a predefined signal or signal pattern, a health condition or disease.

14. The method of claim 12, further comprising:
processing the electronic signal via a neural network or pattern recognition algorithm, wherein the electronic signal correlates with a predefined signal or signal pattern associated with a health condition or disease; and
identifying, based at least in part on the determination of a correlation with a predefined signal or signal pattern, a health condition or disease.

15. The method of claim 12, further comprising:
classifying the electronic signal as a new signal or signal pattern associated with the health condition or disease; and
storing the new pattern in a data storage device.

16. The method of claim 12, further comprising:
facilitating a treatment for the subject to address the health condition or disease.

17. A sensor comprising:
a first sensor component operable to expose one or more nanoparticles to at least one VOC (volatile organic compound) in an exhaled breath of a subject, wherein the one or more nanoparticles are operable to react to a presence of or contact with the at least one VOC (volatile organic compound);
a second sensor component operable to generate an electronic signal when the one or more nanoparticles react to the presence of or contact with the at least one VOC, wherein the electronic signal is associated with a concentration or amount of the at least one VOC; and
an electronic circuit operable to transmit the electronic signal to an output device or computer processor.

18. The sensor of claim 17, wherein the first sensor component comprises at least one of the following: a MXene; a customized form 2D Mn+iXn (Ts) MXene composition; a 2D (two-dimensional) Ti3C2 nanosheet; Ti3C2Tx; Ti₃C₂(OH)₂; Ti₃C₂ MXene; a 3D MXene (3D-M) framework; one or more metal oxide nanoparticles blended with a hybrid structure of graphene; a conducting polymer such as polyaniline (PAni) and polypyrrole (PPY); a supramolecule; a cavitand (macrocyclic compounds based on resorcinarenes); or a relatively thin film of a supramolecule or a calixresorcinarene.

19. The sensor of claim 17, wherein the VOCs comprise at least one of the following: a ketone, acetone, an aldehyde, an acetaldehyde, a hydrocarbon, an alkane, a pentane, an alkene, or a fatty acid.

20. The sensor of claim 17, wherein the one or more nanoparticles comprise a plurality of different nanoparticles, each of the plurality of different nanoparticles selected to trap, bind, or react with a respective different VOC, and wherein the electronic signal comprises respective signal components associated with each of the plurality of different nanoparticles trapping, binding, or reacting with a respective different VOC.
